(19) United States
(12) Patent Application Publication
(10) Pub. No.: US 2023/0349888 A1
(43) Pub. Date: Nov. 2, 2023

(54) A HIGH-THROUGHPUT SCREENING METHOD TO DISCOVER OPTIMAL GRNA PAIRS FOR CRISPR-MEDIATED EXON DELETION

(71) Applicant: Duke University, Durham, NC (US)

(72) Inventors: Charles A. Gersbach, Chapel Hill, NC (US); Veronica Gough, Durham, NC (US); Karen Bulaklak, Durham, NC (US)

(21) Appl. No.: 17/921,332

(22) PCT Filed: Apr. 27, 2021
(86) PCT No.: PCT/US2021/029498

§ 371 Date: Oct. 25, 2022

Related U.S. Application Data
(60) Provisional application No. 63/016,238, filed on Apr. 27, 2020.
(60) Provisional application No. 63/016,204, filed on Apr. 27, 2020.
(60) Provisional application No. 63/023,460, filed on May 12, 2020.

(51) Int. Cl. G01N 33/50; C12N 15/11; C12N 9/22; C12N 15/10; C12N 15/90; C12N 15/85; C07K 14/47; A61K 49/00; A01K 67/027

(52) U.S. Cl. CPC G01N 33/5088; C12N 15/11; C12N 15/111; C12N 9/22; C12N 15/1082; C12N 15/907; C12N 15/8509; C07K 14/4707; A61K 49/0008; A01K 67/0276; C12N 2015/8536; C12N 2310/20; C12N 2800/80; A01K 2267/0306; A01K 2227/105; A01K 2217/056; A01K 2217/072

(57) ABSTRACT

Disclosed herein are methods of using probes for high-throughput screening of guide RNA (gRNA) efficiency for Clustered Regularly Interspaced Short Palindromic Repeats (CRISPR)/CRiSPR-associated (Cas)-based genome editing systems. Further disclosed herein is a humanized transgenic mouse model that recapitulates the severe DMD pathology of human patients. The mouse model may be used for determining the feasibility of CRISPR-based therapies for the correction of the human dystrophin gene by gene editing and methods of use.

### CROSS-REFERENCE TO RELATED APPLICATIONS

This application claims priority to U.S. Provisional Patent Application No. 63/016,238, filed Apr. 27, 2020, U.S. Provisional Patent Application No. 63/016,204, filed Apr. 27, 2020, and U.S. Provisional Patent Application No. 63/023,460, filed May 12, 2020, each of which is incorporated herein by reference in its entirety.

### STATEMENT REGARDING FEDERALLY SPONSORED RESEARCH

This invention was made with government support under grant R01AR069085 awarded by the National Institutes of Health. The government has certain rights in the invention.

### FIELD

This disclosure relates to the field of gene expression alteration, genome engineering, and genomic alteration of genes using Clustered Regularly Interspaced Short Palindromic Repeats (CRISPR)/CRISPR-associated (Cas) 9-based systems and viral delivery systems. The present disclosure also relates to high-throughput screening of CRISPR/Cas9-based systems for identification of high efficiency gRNA pairs. The present disclosure further relates to genetically modified animals and screening agents and use of genetically modified animals for treatment of diseases such as Duchenne muscular dystrophy (DMD).

### INTRODUCTION

Exon skipping or deletion has proven to be a powerful strategy for the correction of genetic diseases, where removal of an exon can correct reading frames distorted by aberrant splicing or other mutations. For example, Duchene muscular dystrophy (DMD), often caused by the deletion of one or more exons in the DMD gene, has a number of promising exon-skipping strategies. Antisense oligonucleotides have been used to force exon skipping during RNA splicing, but the effects are transient and require re-administration. CRISPR-Cas9 can also be directed to cleave the intronic regions on either side of an out-of-frame exon, enabling the non-homologous end joining (NHEJ) repair process to permanently remove the exon from the genome. This strategy of delivering Staphylococcus aureus Cas9 (SaCas9) and two gRNAs with AAV has shown some efficacy in vivo, with exon deletion efficiencies ranging from 2-4% in the heart and skeletal muscle. These DNA editing rates restored dystrophin protein to almost 10% of wild-type levels in skeletal muscle, however, higher protein expression is usually required for an asymptomatic phenotype.

Optimizing gRNA design is key because there is a wide range of on-target activity between different genomic target sites, and the large size of introns (tens to hundreds of kb) provide ample space for finding gRNAs with desirable on- and off-target editing profiles. Previous studies have not taken advantage of this targeting range or sequence diversity, only testing tens of gRNAs. Moreover, previous studies have used high individual gRNA activity to predict optimal gRNA pairs, even though it is established that the context of the gRNA pair is an important parameter in determining genomic deletion efficiency. Further, the accurate measurement of relative deletion efficiencies is difficult—the low, cell type-specific expression of genes such as dystrophin precludes the use of a genetic reporter and PCR-based assays are heavily biased by the wide range of deletion sizes. Thus, there remains a need for a high-throughput screening method to identify novel gRNA pairs that exhibit high efficiency in addition to desirable on- and off-target effects.

### SUMMARY

In an aspect, the disclosure relates to a method of screening for a pair of gRNA molecules for editing a genomic nucleic acid in a subject. The method may include (a) generating a plurality of pairs of gRNA molecules, each pair comprising a first gRNA and a second gRNA, wherein the first gRNA targets a first nucleic acid sequence and the second gRNA targets a second nucleic acid sequence; (b) expressing a Cas9 protein or a fusion protein comprising the Cas9 protein, and the plurality of pairs of gRNA molecules in a plurality of cells, wherein one pair of gRNA molecules is expressed in a cell, and wherein the first gRNA directs the Cas9 protein or fusion protein to cut the first nucleic acid sequence and the second gRNA directs the Cas9 protein or fusion protein to cut the second nucleic acid sequence. In some embodiments, expressing the Cas9 protein or the fusion protein comprising the Cas9 protein, and the plurality of pairs of gRNA molecules in the plurality of cells, wherein one pair of gRNA molecules is expressed in a cell, and wherein the first gRNA directs the Cas9 protein or fusion protein to cut the first nucleic acid sequence and the second gRNA directs the Cas9 protein or fusion protein to cut the second nucleic acid sequence in step (b), thereby forms an excised nucleic acid and a new junction in the genomic nucleic acid. In some embodiments, the excised nucleic acid is in-frame. In some embodiments, the genomic nucleic acid comprises at least one exon of a dystrophin gene, wherein the first nucleic acid sequence comprises a first intron of the dystrophin gene and the second nucleic acid sequence comprises a second intron of the dystrophin gene, and wherein the first intron is adjacent to one side of the at least one exon and the second intron is adjacent to the other side of the at least one exon. In some embodiments, the at least one exon is in between the first and second introns in the genomic nucleic acid. In some embodiments, the genomic nucleic acid comprises two or more exons of a dystrophin gene, wherein the first nucleic acid sequence comprises a first intron of the dystrophin gene and the second nucleic acid sequence comprises a second intron of the dystrophin gene, and wherein the first intron is adjacent to one side of the two or more exons and the second intron is adjacent to the other side of the two or more exons. In some embodiments, the two or more exons are in between the first and second introns in the genomic nucleic acid. In some embodiments, the expression is effected by transfecting the plurality of cells with a plurality of vectors, wherein each cell is transfected with a first vector encoding one pair of gRNA molecules and a second vector encoding the Cas9 protein or fusion protein, wherein each cell is transfected with a different first vector encoding a different pair of gRNA molecules. In some embodiments, the first vector and second vector are each a viral vector. In some embodiments, the viral vector is a lentiviral vector, a AAV vector, or an adenoviral vector. In some embodiments, the method further includes (c) isolating the genomic nucleic acid from the plurality of cells; and/or (d) contacting the genomic nucleic acid with a first pool of probes, wherein one or more different probes specifically bind to each new junction and a portion of the first nucleic acid sequence; and/or (e) isolating the genomic nucleic acid bound to the first pool of probes; and/or (f) contacting the genomic nucleic acid bound to the first pool of probes with a second pool of probes, wherein one or more different probes specifically bind to each new junction and a portion of the second nucleic acid sequence; and/or (g) isolating the genomic nucleic acid bound to the first and second pools of probes; and/or (h) sequencing the isolated genomic nucleic acid bound to the first and second pools of probes; and/or (i) aligning the sequenced isolated genomic nucleic acid to identify the sequenced new junctions; and/or (j) assigning each sequenced new junction to the corresponding pair of gRNA molecules. In some embodiments, step (i) comprises computationally aligning the sequences of the isolated genomic nucleic acid to identify the sequenced new junctions. In some embodiments, the method further includes identifying the pair of gRNA molecules having a greater number of sequenced new junctions as the pair of gRNA molecules having greater efficiency. In some embodiments, the probes each have a length of about 100 bp to about 140 bp. In some embodiments, the excised nucleic acid comprises exon 51 of the dystrophin gene. In some embodiments, the excised nucleic acid comprises exons 45-55 of the dystrophin gene. In some embodiments, the first nucleic acid sequence is within intron 50 of the dystrophin gene. In some embodiments, the second nucleic acid sequence is within intron 51 of the dystrophin gene. In some embodiments, the first nucleic acid sequence is within intron 44 of the dystrophin gene. In some embodiments, the second nucleic acid sequence is within intron 55 of the dystrophin gene. In some embodiments, the probes are biotinylated probes.

In a further aspect, the disclosure relates to a pair of gRNA molecules identified by a method as detailed herein.

Another aspect of the disclosure provides a CRISPR/Cas9 system comprising a pair of gRNA molecules as detailed herein.

Another aspect of the disclosure provides a gRNA molecule that binds and targets a polynucleotide sequence. In some embodiments, the gRNA molecule binds or is encoded by a polynucleotide comprising a sequence selected from SEQ ID NOs: 55-78, or the gRNA molecule comprises a polynucleotide sequence selected from SEQ ID NOs: 79-102.

Another aspect of the disclosure provides a transgenic mouse whose genome comprises: a mutation in the mouse dystrophin gene; a mutant human dystrophin gene on chromosome 5; and a mutation in the mouse utrophin gene. In some embodiments, the mutation in the mouse dystrophin gene comprises an insertion or deletion in the mouse dystrophin gene that prevents protein expression from the mouse dystrophin gene. In some embodiments, the mutation in the mouse dystrophin gene comprises a premature stop codon in exon 23 of the mouse dystrophin gene. In some embodiments, the mutant human dystrophin gene has at least one exon deleted. In some embodiments, the mutant human dystrophin gene has exon 52 deleted. In some embodiments, the mutation in the mouse utrophin gene is a functional deletion of the mouse utrophin gene. In some embodiments, the mutation in the mouse utrophin gene comprises an insertion or deletion in the mouse utrophin gene that prevents protein expression from the mouse utrophin gene. In some embodiments, the mutation in the mouse utrophin gene comprises an insertion in exon 7 of the mouse utrophin gene. In some embodiments, the mutation in the mouse utrophin gene comprises a deletion of the entire mouse utrophin gene. In some embodiments, the mouse is heterozygous for the mutation in the mouse utrophin gene. In some embodiments, the mouse is homozygous for the mutation in the mouse utrophin gene. In some embodiments, the mouse has reduced life span, reduced body mass, reduced body strength, reduced motor coordination, reduced balance, and/or reduced forelimb strength as compared to a wild-type mouse. In some embodiments, the mouse has reduced life span, reduced body mass, reduced body strength, reduced motor coordination, reduced balance, and/or reduced forelimb strength as compared to a control mouse whose genome comprises a wild-type utrophin gene and a mutation in the mouse dystrophin gene. In some embodiments, the mouse has reduced lifespan, reduced body mass, reduced body strength, reduced motor coordination, reduced balance, and/or reduced forelimb strength as compared to a control mouse whose genome comprises a wild-type utrophin gene, a mutation in the mouse dystrophin gene, and a mutant human dystrophin gene. In some embodiments, the mouse has increased muscle damage as compared to (i) a wild-type mouse, (ii) a control mouse whose genome comprises a wild-type utrophin gene and a mutation in the mouse dystrophin gene, and/or (iii) a control mouse whose genome comprises a wild-type utrophin gene, a mutation in the mouse dystrophin gene, and a mutant human dystrophin gene. In some embodiments, the muscle damage comprises one or more of degeneration of the muscle, fibrosis of the muscle, and elevated serum creatine kinase. In some embodiments, the mouse does not exhibit detectable dystrophin protein in heart or skeletal muscle. In some embodiments, the mouse is a hDMDΔ52/mdx/Utrn KO mouse.

Another aspect of the disclosure provides an isolated cell or biological material obtained from a mouse as detailed herein. In some embodiments, the biological material includes a protein, a lipid, a nucleotide, fat, muscle, or a tissue.

Another aspect of the disclosure provides a method of correcting a dystrophin gene mutation. The method may include administering to a mouse as detailed herein a CRISPR/Cas9 gene editing composition. In some embodiments, the CRISPR/Cas9 gene editing composition comprises: (a) at least one guide RNA (gRNA) targeting the mutant human dystrophin gene; and (b) a Cas9 protein or a fusion protein comprising the Cas9 protein. In some embodiments, the CRISPR/Cas9 gene editing composition comprises a first gRNA and a second gRNA, wherein the first gRNA and the second gRNA are configured to form a first and a second double strand break in a first and a second intron flanking exon 51 of the mutant human dystrophin gene, respectively, thereby deleting exon 51. In some embodiments, the CRISPR/Cas9 gene editing composition comprises a first gRNA and a second gRNA, wherein the first gRNA and the second gRNA are configured to form a first and a second double strand break in a first and a second intron flanking exons 45-55 of the mutant human dystrophin gene, respectively, thereby deleting exons 45-55. In some embodiments, the dystrophin gene mutation is corrected in a cell of the mouse, and the cell may be a muscle cell, a satellite cell, or an iPSC/iCM. In some embodiments, the correction restores the reading frame of the human dystrophin gene. In some embodiments, the correction results in expression of an at least partially functional human dystrophin protein.

Another aspect of the disclosure provides a gamete produced by a mouse as detailed herein. In some embodiments, the gamete does not encode a functional mouse dystrophin protein or a functional mouse utrophin protein.

Another aspect of the disclosure provides an isolated mouse cell, or a progeny cell thereof, isolated from a mouse as detailed herein.

Another aspect of the disclosure provides a primary cell culture or a secondary cell line derived from a mouse as detailed herein.

Another aspect of the disclosure provides a tissue or organ explant or culture thereof, derived from a mouse as detailed herein.

Another aspect of the disclosure provides method of screening therapeutic agents for treating Duchenne muscular dystrophy (DMD). The method may include administering to a mouse as detailed herein one or more therapeutic agents. In some embodiments, the one or more therapeutic agents comprises a small molecule, anti-sense RNA, vector, CRISPR/Cas gene editing system, or biological agent, or a combination thereof. In some embodiments, the vector is a viral vector encoding a gene of interest. In some embodiments, the viral vector is an AAV vector. In some embodiments, the mouse after administration of the one or more therapeutic agents exhibits increased lifespan, reduced body mass, increased body strength, increased motor coordination, increased balance, increased forelimb strength, reduced muscle injury, and/or reduced CK level compared to before administration of the one or more therapeutic agents. In some embodiments, the mouse after administration of the one or more therapeutic agents exhibits increased expression of a dystrophin gene as compared to before administration of the one or more therapeutic agents. In some embodiments, the dystrophin gene is a truncated human dystrophin gene. In some embodiments, the truncated human dystrophin gene comprises a plurality of deletions relative to a wild-type human dystrophin gene. In some embodiments, at least one of the deletions is in exon 52.

The disclosure provides for other aspects and embodiments that will be apparent in light of the following detailed description and accompanying figures.

### BRIEF DESCRIPTION OF THE DRAWINGS

FIG. 1A, FIG. 1B, FIG. 1C, and FIG. 1D are schematics for the method of screening a pool of gRNA pairs for exon deletion. FIG. 1A shows the design of gRNAs. FIG. 1B shows the transduction of a stable saCas9 cell line with a lentiviral gRNA pair library. FIG. 1C shows the harvest gDNA, library prep, and enrichment for junctions with biotinylated probes. FIG. 1D shows the sequence junctions to determine the frequency of each unique junction created after a deletion event.

FIG. 2 is a schematic of the biotinylated probe design.

FIG. 3 shows an experiment using the enrichment and sequencing method on cells that only received a single gRNA pair.

FIG. 4 shows the frequency with which deletion-making gRNA pairs were identified by sequencing. The frequency with which deletion-making gRNA pairs were identified by sequencing was normalized by initial gRNA abundance and bias introduced by probe hybridization. For all 2.080 pairs shown, many were not detected, but several pairs were detected with high frequency.

FIG. 5 shows the top 25 pairs of gRNAs identified by sequencing. The gray bar indicates a previously used gRNA pair that was identified with a conventional low-throughput method. Each data point represents the value from a replicate, n=4.

FIG. 6 is a schematic of the breeding pair and step 1 of the breeding scheme.

FIG. 7 is a schematic of steps 2-4 of the breeding scheme.

FIG. 8A, FIG. 88, FIG. 8C, and FIG. 8D show whole body strength, balance, and motor coordination of the various dystrophin and utrophin genotypes using the rotarod test. FIG. 8A is rotarod performance at the 8-week timepoint. FIG. 8B is rotarod performance at the 12-week timepoint. FIG. 8C is rotarod performance at the 16-week timepoint. FIG. 8D is rotarod performance from 6 to 24 weeks. N=12 for each group, except Utrn KO (N=4-12 depending on age). *P<0.05, **P<0.001, ***P<0.0001, ****P<0.00001.

FIG. 9A, FIG. 9B, and FIG. 9C show forelimb grip strength of the various dystrophin and utrophin genotypes using the grip-strength test. FIG. 9A is grip force performance at the 8-week timepoint. FIG. 9B is grip force performance at the 12-week timepoint. FIG. 9C is grip force performance at the 16-week timepoint. N=12 for 8 and 12 weeks. N=4-12 at 16 weeks. *P<0.05, **P<0.001, ***P<0.0001, ****P<0.00001. Results from a rotarod assay are shown in FIG. 9D. T compares with Utrn (+/+), and #compares with Utrn (+/−).

FIG. 10A and FIG. 10B show body and muscle mass measurements of the various dystrophin and utrophin genotypes. FIG. 10A is body mass from 6 to 24 weeks. FIG. 10B is muscle mass at 24 weeks. N=12 for each group, except Utrn KO (N=4-12 depending on age). ***P<0.0001.

FIG. 11 shows survival over 24 weeks of the various dystrophin and utrophin genotypes. N=12 for each group at start.

FIG. 12A, FIG. 12B, FIG. 12C, and FIG. 12D show H&E staining of diaphragm muscle to assess dystrophic pathology in the various dystrophin and utrophin genotypes at 24 weeks of age. FIG. 12A is the hDMD/mdx genotype. FIG. 12B is the hDMDΔ52/mdx genotype. FIG. 12C is the hDMDΔ52/mdx/Utrn het genotype. FIG. 12D is the hDMDΔ52/mdx/Utrn KO genotype. Images are 10× magnification.

FIG. 13A, FIG. 13B, FIG. 13C, and FIG. 13D show Masson trichrome staining of diaphragm muscle to assess fibrosis in the various dystrophin and utrophin genotypes at 24 weeks of age. FIG. 13A is the hDMD/mdx genotype. FIG. 13B is the hDMDΔ52/mdx genotype. FIG. 13C is the hDMDΔ52/mdx/Utrn het genotype. FIG. 13D is the hDMDΔ52/mdx/Utrn KO genotype. Images are 10× magnification.

FIG. 14A shows levels of creatine kinase in the serum of the various dystrophin and utrophin genotypes at 24 weeks of age. N=4-8 for each group. *P<0.05, **P<0.0001 compared to hDMD/mdx. FIG. 14B shows the body mass of the various dystrophin and utrophin genotypes at 24 weeks of age. FIG. 14C shows percent survival of the various dystrophin and utrophin genotypes at 24 weeks of age.

FIG. 15 is a schematic of CRISPR/Cas9 treatment of the various dystrophin and utrophin mice.

FIG. 16 shows PCR (top) and Western blot (bottom) of utrophin heterozygous and homozygous knockout mice. CRISPR/Cas9 treatment restores dystrophin reading frame and protein expression. 3.125 μg of protein lysate was loaded for the hDMD/mdx positive control. One mouse per treatment group per genotype is represented.

FIG. 17A and FIG. 17B show immunofluorescent staining of hDMDΔ52/mdx/Utrn het neonate mice treated with CRISPR/Cas9. FIG. 17A is the AAV9-control CRISPR/Cas9. FIG. 17B is the AAV9-ΔExon 51 CRISPR. Red is dystrophin and blue is DAPI. Images are 10× magnification, tissue is from mice 8 weeks of age.

FIG. 18A and FIG. 18B show immunofluorescent staining of hDMDΔ52/mdx/Utrn KO neonate mice treated with CRISPR/Cas9. FIG. 18A is the AAV9-control CRISPR/Cas9. FIG. 18B is the AAV9-ΔExon 51 CRISPR. Red is dystrophin and blue is DAPI. Images are 10× magnification, tissue is from mice 8 weeks of age. FIG. 18C is a graph showing increased serum creatine kinase (CK) after CRISPR-ΔExon 51 treatment in both hDMDΔ52/mdx/Utrn het and hDMDΔ52/mdx/Utrn KO mice.

FIG. 19A, FIG. 19B, and FIG. 19C show immunofluorescent staining of the tibialis anterior of hDMDΔ52/mdx/Utrn KO adult mice treated with CRISPR/Cas9. FIG. 19A is hDMD/mdx mice untreated. FIG. 19B is hDMDΔ52/mdx/Utrn KO mice treated with AAV9-control CRISPR/Cas9. FIG. 19C is hDMDΔ52/mdx/Utrn KO mice treated with AAV9-ΔExon 51 CRISPR. Red is dystrophin and blue is DAPI. Images are 10× magnification, tissue is from mice 16 weeks of age. FIG. 19D is a graph of percent dystrophin positive fibers in various tissues. Dystrophin positive fibers were quantified using 5 images per mouse, N=3 per group. FIG. 19E is a survival curve representing each treatment group showing exon 51 deletion improved survival in Utrn KO mice. Log-rank test was performed. N=4-6 per group, *P=0.0251. FIG. 19F is a graph showing exon 51 deletion improved motor function in Utrn KO mice. Forelimb grip strength was measured at 10 weeks and normalized to body weight. N=3-4 per group.

### DETAILED DESCRIPTION

As described herein, certain methods and engineered gRNAs have been discovered to be useful with CRISPR/CRISPR-associated (Cas) 9-based gene editing systems for altering the expression (i.e., genome engineering) and correcting or reducing the effects of mutations in the dystrophin gene involved in genetic diseases such as DMD. The disclosed high-throughput CRISPR/Cas9 gRNA screening method was generated to yield novel junctions that are more amenable to clinical translation. For example, introns of the dystrophin gene are large and many different sequences within the introns can be targeted with gRNAs. gRNAs targeting each intronic target sequence have varying on- and off-target effects that need to be optimized. The disclosed method provides a process to screen thousands of gRNA pairs to identify gRNA pairs that mediate high efficiency exon deletion with few to no off-target effects. Since each gRNA pair yields a unique junction that is created after a deletion event, the frequency of each junction is a direct measure of the deletion efficiency for a gRNA pair. The gRNAs identified by the disclosed method, which target human dystrophin gene sequences, can be used with the CRISPR/Cas9-based system to target regions of the human dystrophin gene, such as exon 51, causing genomic deletions of this exon in order to restore expression of functional dystrophin in cells of DMD patients. The method provides a means of identifying gRNA pairs that are effective, efficient, and facilitate successful genome modification, as well as provide a means to rewrite the human genome for therapeutic applications and target model species for basic science applications.

In addition, the screening method may comprise an enrichment and sequencing method that can be used to detect unique intron-intron junctions as well as detect perfect ligation of gRNA cut-sites. The method may also be used to quantify the level of exon deletion made by each gRNA pair. The screening method relies only on genomic DNA as an output and does not require a reporter. Therefore, the method can be applied on any locus in any cell type. Thus, it can be easily adapted to optimize gRNA pairs for any genetic disease where a targeted deletion is a viable therapeutic strategy.

Further disclosed herein is a mouse model to better recapitulate the human DMD phenotype. To exacerbate dystrophic pathology in animal models, many different approaches have been taken, including chemical treatment and various genetic knockouts. The utrophin protein, which is normally expressed in the neuromuscular junction, shares functional domains with dystrophin. Overexpression of utrophin has resulted in muscle membrane localization, similar to dystrophin, and functional improvements in dystrophic animal models. Utrophin may compensate for dystrophin. The humanized mouse model detailed herein includes a dystrophin and utrophin double knockout.

The disclosed mouse model improves clinical translation of therapeutics tested in mouse models. For example, mouse models that express a wild-type utrophin gene and a mutation in the mouse dystrophin gene or a mutation in the mouse dystrophin gene and a mutant human dystrophin gene display a mild DMD pathology and phenotype. In contrast, the disclosed mouse models do not express utrophin or dystrophin. hDMDΔ52/mdx mice were crossed with mice lacking the murine utrophin gene to generate hDMDΔ52/mdx/Utrn KO mice. The animal models and methods detailed herein may be useful for studying genetic diseases, such as DMD, and altering expression of dystrophin and utrophin using gene editing systems, such as CRISPR-Cas9. The disclosed mouse model can be used to assess the efficacy of therapeutics using phenotypic measurements such as motor function and lifespan.

Also described herein are methods for delivering CRISPR/Cas9-based gene editing systems and multiple gRNAs to target the human dystrophin gene. The CRISPR/Cas9-based gene editing system can be delivered using an AAV vector, including modified AAV vectors. Provided herein are ways to deliver this class of therapeutics to mouse models that is effective, efficient, and facilitates successful genome modification, as well as provide a means to rewrite the human genome for therapeutic applications and target animal models for basic science applications. The methods may relate to the use of a single AAV vector for the delivery of all of the editing components necessary for the excision of exon 51 of dystrophin.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by the CRISPR/Cas-based gene editing system.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be an subject or cell without an agonist as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3,500 amino acids.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal

“Genome editing” or “gene editing” as used herein refers to changing the DNA sequence of a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

The term “heterozygous” as used herein refers to a subject comprising two different alleles for a particular gene.

The term “homozygous” as used herein refers to a subject comprising two identical alleles for a particular gene.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Junction” as used herein refers to a point in a nucleic acid where one or more nucleic acids are joined. In some embodiments, a junction may be a point in a nucleic acid where an intron is joined to an exon. In some embodiments, a junction may be a point in a nucleic acid where an intron or portion thereof is joined to itself or a different intron or portion thereof. In some embodiments, a junction may be a point in a nucleic acid where double-strand breaks occurred in the nucleic acid.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair.

These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynudeotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynudeotide can contain combinations of deoxyribo- and ribo-nucleotides, and combinations of bases including, for example, uracil, adenine, thymine, cytosine, guanine, inosine, xanthine hypoxanthine, isocytosine, and isoguanine.

Polynucleotides can be obtained by chemical synthesis methods or by recombinant methods. A genomic nucleic acid can be genomic DNA where it is chromosomal DNA of an organism, the organism includes cell lines commonly used in research such as HEK293T cells.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Skeletal muscle” as used herein refers to a type of striated muscle, which is under the control of the somatic nervous system and attached to bones by bundles of collagen fibers known as tendons. Skeletal muscle is made up of individual components known as myocytes, or “muscle cells”, sometimes colloquially called “muscle fibers.” Myocytes are formed from the fusion of developmental myoblasts (a type of embryonic progenitor cell that gives rise to a muscle cell) in a process known as myogenesis. These long, cylindrical, multinucleated cells are also called myofibers.

“Skeletal muscle condition” as used herein refers to a condition related to the skeletal muscle, such as muscular dystrophies, aging, muscle degeneration, wound healing, and muscle weakness or atrophy.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated. In certain embodiments, the target gene is a gene involved in DMD.

“Target region” as used herein refers to the region of the target gene to which the CRISPR/Cas9-based gene editing or targeting system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “treatment” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease. As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome, or yeast artificial chromosome. A vector may be a DNA or RNA vector. A vector may be a self-replicating extrachromosomal vector, and preferably, is a DNA plasmid. For example, the vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Dystrophin Gene

Dystrophin is a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane. The dystrophin gene is 2.2 megabases at locus Xp21. The primary transcription measures about 2,400 kb with the mature mRNA being about 14 kb. 79 exons include approximately 2.2 million nucleotides and code for the protein which is over 3,500 amino acids. Normal skeleton muscle tissue contains only small amounts of dystrophin, but its absence of abnormal expression leads to the development of severe and incurable symptoms. Some mutations in the dystrophin gene lead to the production of defective dystrophin and severe dystrophic phenotype in affected patients. Some mutations in the dystrophin gene lead to partially-functional dystrophin protein and a much milder dystrophic phenotype in affected patients.

DMD is the result of inherited or spontaneous X-linked recessive mutation(s) that cause nonsense or frame shift mutations in the dystrophin gene. DMD is a severe, highly debilitating and incurable muscle disease. DMD is the most prevalent lethal heritable childhood disease and affects approximately one in 5,000 newborn males. DMD is characterized by muscle deterioration, progressive muscle weakness, often leading to mortality in subjects at age mid-twenties, due to the lack of a functional dystrophin gene, and premature death. Most mutations are deletions in the dystrophin gene that disrupt the reading frame. Naturally occurring mutations and their consequences are relatively well understood for DMD. In-frame deletions that occur in the exon 45-55 regions contained within the rod domain can produce highly functional dystrophin proteins, and many carriers are asymptomatic or display mild symptoms. Exons 45-55 of dystrophin are a mutational hotspot. Furthermore, more than 60% of patients may be treated by targeting exons in this region of the dystrophin gene. Efforts have been made to restore the disrupted dystrophin reading frame in DMD patients by skipping non-essential exon(s) (e.g., exon 45 skipping) during mRNA splicing to produce internally deleted but functional dystrophin proteins. The deletion of internal dystrophin exon(s) (for example, deletion of exon 45) may retain the proper reading frame and can generate an internally truncated but partially functional dystrophin protein. Deletions between exons 45-55 of dystrophin can result in a phenotype that is much milder compared to DMD.

A dystrophin gene may be a mutant dystrophin gene. A dystrophin gene may be a wild-type dystrophin gene. A dystrophin gene can be a mammal dystrophin gene. In some embodiments, a dystrophin gene is a dog dystrophin gene. In some embodiments, the dystrophin gene is a rat dystrophin gene. In some embodiments, the dystrophin gene is a mouse dystrophin gene. In some embodiments, the dystrophin gene is a human dystrophin gene. A dystrophin gene may have a sequence that is functionally identical to a wild-type dystrophin gene, for example, the sequence may be codon-optimized but still encode for the same protein as the wild-type dystrophin. A mutant dystrophin gene may include one or more mutations relative to the wild-type dystrophin gene. Mutations may include, for example, nucleotide deletions, substitutions, additions, transversions, or combinations thereof. A mutation in the dystrophin gene may be a functional deletion of the dystrophin gene. In some embodiments, the mutation in the dystrophin gene comprises an insertion or deletion in the dystrophin gene that prevents protein expression from the dystrophin gene. Mutations may be in one or more exons and/or introns. Mutations may include deletions of all or parts of at least one intron and/or exon. An exon of a mutant dystrophin gene may be mutated or at least partially deleted from the dystrophin gene. An exon of a mutant dystrophin gene may be fully deleted. A mutant dystrophin gene may have a portion or fragment thereof that corresponds to the corresponding sequence in the wild-type dystrophin gene. In some embodiments, a disrupted dystrophin gene caused by a deleted or mutated exon can be restored in DMD patients by adding back the corresponding wild-type exon. In some embodiments, disrupted dystrophin caused by a deleted or mutated exon 52 can be restored in DMD patients by adding back in wild-type exon 52. In certain embodiments, addition of exon 52 to restore reading frame ameliorates the phenotype in DMD subjects, including DMD subjects with deletion mutations. In certain embodiments, one or more exons may be added and inserted into the disrupted dystrophin gene. The one or more exons may be added and inserted so as to restore the corresponding mutated or deleted exon(s) in dystrophin. The one or more exons may be added and inserted into the disrupted dystrophin gene in addition to adding back and inserting the exon 52. In certain embodiments, exon 52 of a dystrophin gene refers to the 52nd exon of the dystrophin gene. Exon 52 is frequently adjacent to frame-disrupting deletions in DMD patients. The mutation in the mouse dystrophin gene may comprise an insertion or deletion in the mouse dystrophin gene that prevents protein expression from the mouse dystrophin gene. In some embodiments, a disrupted dystrophin gene may be caused by a mutation in exon 23 of the mouse dystrophin gene. In some embodiments, the mutation in the mouse dystrophin gene comprises a premature stop codon in exon 23 of the mouse dystrophin gene. In some embodiments, the mutation in the human dystrophin gene includes deletion of at least one exon. In some embodiments, the mutant human dystrophin gene has exon 52 deleted.

### 3. Utrophin Gene

Utrophin is a large multidomain protein which is a part of a family of actin-binding proteins that includes dystrophin. Utrophin is a homolog of dystrophin. Utrophin is expressed in developing muscle and is enriched at the neuromuscular junction in mature muscle. Utrophin levels are decreased as the myofibers mature and is replaced by dystrophin. Similar to dystrophin, utrophin interacts with the dystrophin-associated protein complex. The protein complex that connects the cytoskeleton of a muscle fiber to the surrounding extracellular matrix through the cell membrane. The utrophin gene is about 900 kb at locus 6q24.2 in humans and locus 10 A1-A2; 10 3.77 cM in mice (SEQ ID NO: 37). In humans, 85 exons encode for the protein which is 3,433 amino acids. In mice, 81 exons encode for the protein which is 3,430 amino acids (SEQ ID NO: 38). Mature skeletal muscle tissue contains utrophin, but its absence or abnormal expression does not result in any physical or behavioral deficits. Mutations in the utrophin gene lead to the production of defective utrophin and in combination with mutations in the dystrophin gene results in a severe dystrophic phenotype.

A utrophin gene may be a mutant utrophin gene. A utrophin gene may be a wild-type utrophin gene. A utrophin gene may have a sequence that is functionally identical to a wild-type utrophin gene, for example, the sequence may be codon-optimized but still encode for the same protein as the wild-type utrophin. A mutant utrophin gene may include one or more mutations relative to the wild-type utrophin gene. Mutations may include, for example, nucleotide deletions or truncations, substitutions, additions, transversions, or combinations thereof. A mutation in the mouse utrophin gene may be a functional deletion of the mouse utrophin gene. In some embodiments, the mutation in the mouse utrophin gene comprises an insertion or deletion in the mouse utrophin gene that prevents protein expression from the mouse utrophin gene. For example, the mutation in the mouse utrophin gene may include an insertion in exon 7 of the mouse utrophin gene. Such an insertion in exon 7 may prevent protein expression from the mouse utrophin gene. Mutations may include deletions of all or parts of at least one intron and/or exon. An exon of a mutant utrophin gene may be mutated or at least partially deleted from the utrophin gene. An exon of a mutant utrophin gene may be fully deleted. The mutation in the mouse utrophin gene may be a deletion of the entire mouse utrophin gene. A mutant utrophin gene may have a portion or fragment thereof that corresponds to the corresponding sequence in the wild-type utrophin gene. In some embodiments, disrupted utrophin is caused by an insertion of a neomycin cassette into exon 7 of the mouse utrophin gene. In certain embodiments, one or more exons may be added and inserted into the disrupted utrophin gene.

### 4. Transgenic Mouse

Previous gene replacement strategies to treat DMD utilizing miniaturized dystrophin transgenes delivered via recombinant adeno-associated viruses (rAAVs) have been successful in ameliorating dystrophic pathology to create a Becker muscular dystrophy (BMD)-like phenotype. Still, accelerated muscle turnover, an immune response to the foreign dystrophin protein, and packaging constraints of rAAVs are significant barriers to achieving maximal therapeutic efficacy for DMD. CRISPR/Cas9 is a promising strategy for genome editing and allows permanent, targeted excision of defective dystrophin exons. CRISPR/Cas9 has been previously used in dystrophic mdx mice via AAV delivery to excise exons and restore the dystrophin reading frame, producing a shorter, yet functional protein. As a result, skeletal muscle pathology and function were improved. In order to test the efficacy of CRISPR-mediated treatment strategies targeting the human dystrophin gene, humanized mouse model, hDMDΔ52/mdx, has previously been generated. These mice contain a deletion of exon 52 in the human dystrophin gene located in chromosome 5 creating an out-of-frame mutation, as well as a point mutation in exon 23 of the mouse dystrophin gene. As a result, these mice are completely dystrophin-null and display mild muscle pathology and respiratory function deficits compared to wild-type mice. While CRISPR treatment in hDMDΔ52/mdx mice can restore the human dystrophin reading frame and allow protein expression, functional improvements are difficult to discern due to their mild pathology and phenotype at baseline. Furthermore, utilizing a mouse model that better reflects the severity of DMD patient symptoms would be highly informative in determining the feasibility of CRISPR-based therapies. There has been a need for a model to effectively recapitulate the human DMD phenotype and to assess and develop future human-targeted therapies for DMD.

Provided herein is a new transgenic mouse. The genome of the transgenic mouse may include a mutation in the mouse dystrophin gene. For example, the genome of the transgenic mouse may include a premature stop codon in exon 23 of the mouse dystrophin gene. Insertion of a premature stop codon in exon 23 of the mouse dystrophin gene may result in a functional knockout of the mouse dystrophin gene in the mouse. The genome of the transgenic mouse may include a wild-type human dystrophin gene. The genome of the transgenic mouse may include a mutant human dystrophin gene. The mutant human dystrophin gene may be present on chromosome 5 of the mouse. The mutant human dystrophin gene in the genome of the mouse may have an exon deleted, such as, for example, deletion of exon 52.

The genome of the transgenic mouse may include a mutation in the mouse utrophin gene, as detailed above. For example, the genome of the transgenic mouse may include a full or partial or functional deletion of the mouse utrophin gene. The mouse utrophin gene may be fully, partially, or functionally deleted from chromosome 10 of the mouse. The mouse utrophin gene may comprise a polynucleotide of SEQ ID NO: 37. The mouse utrophin gene may encode a polypeptide comprising and amino acid sequence of SEQ ID NO: 38. The mouse may express a human form of a wild-type or mutant dystrophin gene, but may not express mouse utrophin or mouse dystrophin. In some embodiments, the mouse is heterozygous for the mutation in the mouse utrophin gene. In some embodiments, the mouse is homozygous for the mutation in the mouse utrophin gene. The transgenic mouse may be referred to as hDMDΔ52/mdx/UtrnKO.

The transgenic mouse may mirror many aspects of the dystrophic phenotype. The transgenic mouse may display a more severe phenotype as compared to a control mouse. In some embodiments, the mouse has reduced life span, reduced body mass, reduced body strength, reduced motor coordination, reduced balance, respiratory defects, skeletal muscle fibrosis, elevated creatine kinase (CK) levels, and/or reduced forelimb strength as compared to a wild-type mouse. In some embodiments, the mouse has reduced life span, reduced body mass, reduced body strength, reduced motor coordination, reduced balance, respiratory defects, skeletal muscle fibrosis, elevated creatine kinase (CK) levels, and/or reduced forelimb strength as compared to a control mouse whose genome comprises a wild-type utrophin gene and a mutation in the mouse dystrophin gene. In some embodiments, the mouse has reduced lifespan, reduced body mass, reduced body strength, reduced motor coordination, reduced balance, and/or reduced forelimb strength as compared to a control mouse whose genome comprises a wild-type utrophin gene, a mutation in the mouse dystrophin gene, and a mutant human dystrophin gene. In some embodiments, the mouse has increased muscle damage as compared to (i) a wild-type mouse, (ii) a control mouse whose genome comprises a wild-type utrophin gene and a mutation in the mouse dystrophin gene, and/or (iii) a control mouse whose genome comprises a wild-type utrophin gene, a mutation in the mouse dystrophin gene, and a mutant human dystrophin gene. Muscle damage may include one or more of degeneration of the muscle, fibrosis of the muscle, and elevated serum creatine kinase. In some embodiments, the mouse does not exhibit detectable dystrophin protein in heart or skeletal muscle.

Further provided herein is an isolated cell obtained from the mouse, or a progeny cell thereof. The cell may be, for example, a muscle cell, a satellite cell, or an iPSC/iCM. Further provided herein is a gamete produced by the mouse. The gamete may not encode a functional mouse dystrophin protein or a functional mouse utrophin protein. Further provided herein is a primary cell culture or a secondary cell line derived from the mouse, and/or a tissue or organ explant or culture thereof, derived from the mouse.

As detailed above, the transgenic mouse may be used as a mouse model to better recapitulate the human DMD phenotype. The more severe phenotype of the transgenic mouse may enable the improved detection of functional improvements to the mouse, such as, for example, improvements elicited upon administration of a CRISPR/Cas9-based gene editing system detailed herein. For example, the transgenic mouse may be useful for studying genetic diseases, such as DMD, and altering expression of dystrophin and utrophin using the CRISPR/Cas9-based gene editing systems. The disclosed mouse model can also be used to assess the efficacy of therapeutics using phenotypic measurements such as motor function and lifespan.

### 5. CRISPR/Cas9-Based Gene Editing System

The compositions and methods detailed herein may be suitable for any gene editing system or tool wherein one or two, or one or more, targeting nucleases are combined to create a deletion in a genome. Gene editing systems may include, for example, those comprising homing endonucleases, zinc finger nucleases (ZFNs), transcription activator-like effector (TALE) nucleases (TALENs), and clustered regularly interspaced short palindromic repeats (CRISPR)-CRISPR-associated protein (Cas protein) such as Cas9. Homing endonucleases generally cleave their DNA substrates as dimers and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. In some embodiments, the compositions and methods detailed herein may be used with CRISPR/Cas9-based gene editing systems.

Provided herein are CRISPR/Cas9-based gene editing systems. The CRISPR/Cas9-based gene editing system may be used to delete an exon in the dystrophin gene. In certain embodiments, the CRISPR/Cas9-based gene editing system may be used to delete exon 51 in the human dystrophin gene. The CRISPR/Cas9-based gene editing system may include at least one Cas9 protein or a fusion protein, and at least one gRNA. In some embodiments, the mouse models detailed herein are suitable for use with a CRISPR/Cas9-based gene editing system.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas9 forms a complex with the 3′ end of the sgRNA (which may be referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and Ill effector systems) are known. The Type II effector system carries out targeted DNA double-strand break in four sequential steps, using a single effector enzyme, Cas9, to cleave dsDNA. Compared to the Type I and Type Ill effector systems, which require multiple distinct effectors acting as a complex, the Type II effector system may function in alternative contexts such as eukaryotic cells. The Type 11 effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase Ill. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements.

An engineered form of the Type II effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase Ill and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

a. Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., Cycliphilus denitrincans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamenvorans, Pasteurella multocida, Phascolarctobacterium succinatutens, Ralstonia syzygii, Rhodopseudomonas palustris, Rhodovulum sp., Simonsiella mueller, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 18. In certain embodiments, the Cas9 molecule is a Staphylococcus aureus Cas9 molecule (also referred herein as “SaCas9”). SaCas9 may comprise an amino acid sequence of SEQ ID NO: 19.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G) (SEQ ID NO: 7), NNGRRN (R=A or G) (SEQ ID NO: 8), NNGRRT (R=A or G) (SEQ ID NO: 9), or NNGRRV (R=A or G) (SEQ ID NO: 10). In the aforementioned embodiments, N can be any nucleotide residue, for example, any of A, G, C, or T.

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 39).

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 20. A S. pyogenes Cas9 protein with D10A and H849A mutations may comprise an amino acid sequence of SEQ ID NO: 21. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 22. In certain embodiments, the mutant S. aureus Cas9 molecule comprises a N580A mutation. The nucleotide sequence encoding this mutant S. aureus Cas9 molecule is set forth in SEQ ID NO: 23.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 24. Exemplary codon optimized nucleic acid sequences encoding a Cas9 molecule of S. aureus, and optionally containing nuclear localization sequences (NLSs), are set forth in SEQ ID NOs: 25-31. Another exemplary codon optimized nucleic acid sequence encoding a Cas9 molecule of S. aureus comprises the nucleotides 1293-4451 of SEQ ID NO: 32.

b. Cas9 Fusion Protein

Alternatively or additionally, the CRISPR/Cas9-based gene editing system can include a fusion protein. The fusion protein can comprise two heterologous polypeptide domains. The first polypeptide domain comprises a Cas9 protein or a mutated Cas9 protein. The first polypeptide domain is fused to at least one second polypeptide domain. The second polypeptide domain has a different activity than what is endogenous to Cas9 protein. For example, the second polypeptide domain may have an activity such as transcription activation activity, transcription repression activity, transcription release factor activity, histone modification activity, nuclease activity, nucleic acid association activity, methylase activity, demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser)n, wherein n is an integer between 0 and 10 (SEQ ID NO: 40). In a GS linker, n can be adjusted to optimize the linker length and achieve appropriate separation of the functional domains. Other examples of linkers may include, for example, Gly-Gly-Gly-Gly-Gly (SEQ ID NO: 41), Gly-Gly-Ala-Gly-Gly (SEQ ID NO: 42), Gly/Ser rich linkers such as Gly-Gly-Gly-Gly-Ser-Ser-Ser (SEQ ID NO: 43), or Gly/Ala rich linkers such as Gly-Gly-Gly-Gly-Ala-Ala-Ala (SEQ ID NO: 44).

i) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, and/or p300. For example, the fusion protein may comprise dCas9-p300. In some embodiments, p300 comprises a polypeptide having the amino acid sequence of SEQ ID NO: 33 or SEQ ID NO: 34. In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 35, encoded by the polynucleotide of SEQ ID NO: 36.

ii) Transcription Repression Activity

The second polypeptide domain can have transcription repression activity. Non-limiting examples of repressors include Kruppel associated box activity such as a KRAB domain or KRAB, MECP2, EED, ERF repressor domain (ERD), Mad mSIN3 interaction domain (SID) or Mad-SID repressor domain, SID4X repressor domain, Mxil repressor domain, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain has a KRAB domain activity, ERF repressor domain activity, Mxil repressor domain activity, SID4X repressor domain activity, Mad-SID repressor domain activity, DNMT3A or DNMT3L or fusion thereof activity, LSD1 histone demethylase activity, or TATA box binding protein activity. In some embodiments, the polypeptide domain comprises KRAB. For example, the fusion protein may be S. pyogenes dCas9-KRAB (polynucleotide sequence SEQ ID NO: 45; protein sequence SEQ ID NO: 46). The fusion protein may be S. aureus dCas9-KRAB (polynucleotide sequence SEQ ID NO: 47; protein sequence SEQ ID NO: 48).

iii) Transcription Release Factor Activity

The second polypeptide domain can have transcription release factor activity. The second polypeptide domain can have eukaryotic release factor 1 (ERF1) activity or eukaryotic release factor 3 (ERF3) activity.

iv) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 33 or SEQ ID NO: 34.

v) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

vi) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

vii) Methylase Activity

The second polypeptide domain can have methylase activity, which involves transferring a methyl group to DNA, RNA, protein, small molecule, cytosine, or adenine. In some embodiments, the second polypeptide domain includes a DNA methyltransferase.

viii) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1, also known as Tet1CD (Ten-eleven translocation methylcytosine dioxygenase 1; polynucleotide sequence SEQ ID NO: 49; amino acid sequence SEQ ID NO: 50). In some embodiments, the second polypeptide domain has histone demethylase activity. In some embodiments, the second polypeptide domain has DNA demethylase activity.

c. Guide RNA (gRNA)

The CRISPR/Cas-based gene editing system includes at least one gRNA molecule. For example, the CRISPR/Cas-based gene editing system may include two gRNA molecules. The at least one gRNA molecule can bind and recognize a target region. The gRNA provides the targeting of a CRISPR/Cas9-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 52 (RNA), which is encoded by a sequence comprising SEQ ID NO: 51 (DNA). The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target sequence or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The gRNA may target a region near exon 51 of the human dystrophin gene. The gRNA may target a region within intron 50 of the human dystrophin gene. The gRNA may target a region within intron 51 of the human dystrophin gene. The gRNA may bind and target and/or hybridize to a polynucleotide sequence comprising at least one of SEQ ID NOs: 55-78, or a complement thereof, or a variant thereof, or a truncation thereof. The gRNA may be encoded by a polynucleotide sequence comprising at least one of SEQ ID NOs: 55-78, or a complement thereof, or a variant thereof, or a truncation thereof. The gRNA may comprise a polynucleotide sequence of at least one of SEQ ID NOs: 79-102, or a complement thereof, or a variant thereof, or a truncation thereof. A truncation may be 1, 2, 3, 4, 5, 6, 7, 8, or 9 nucleotides shorter than the reference sequence. A truncation may be 1, 2, 3, 4, 5, 6, 7, 8, or 9 nucleotides shorter than the sequence of any one of SEQ ID NOs: 55-78. A truncation may be 1, 2, 3, 4, 5, 6, 7, 8, or 9 nucleotides shorter than the sequence of SEQ ID NO: 55. A truncation may be 1, 2, 3, 4, 5, 6, 7, 8, or 9 nucleotides shorter than the sequence of SEQ ID NO: 56. A truncation may be 1, 2, 3, 4, 5, 6, 7, 8, or 9 nucleotides shorter than the sequence of SEQ ID NO: 57. A truncation may be 1, 2, 3, 4, 5, 6, 7, 8, or 9 nucleotides shorter than the sequence of SEQ ID NO: 58. A truncation may be 1, 2, 3, 4, 5, 6, 7, 8, or 9 nucleotides shorter than the sequence of SEQ ID NO: 59. A truncation may be 1, 2, 3, 4, 5, 6, 7, 8, or 9 nucleotides shorter than the sequence of SEQ ID NO: 60. A truncation may be 1, 2, 3, 4, 5, 6, 7, 8, or 9 nucleotides shorter than the sequence of SEQ ID NO: 61. A truncation may be 1, 2, 3, 4, 5, 6, 7, 8, or 9 nucleotides shorter than the sequence of SEQ ID NO: 62. A truncation may be 1, 2, 3, 4, 5, 6, 7, 8, or 9 nucleotides shorter than the sequence of SEQ ID NO: 63. A truncation may be 1, 2, 3, 4, 5, 6, 7, 8, or 9 nucleotides shorter than the sequence of SEQ ID NO: 64. A truncation may be 1, 2, 3, 4, 5, 6, 7, 8, or 9 nucleotides shorter than the sequence of SEQ ID NO: 65. A truncation may be 1, 2, 3, 4, 5, 6, 7, 8, or 9 nucleotides shorter than the sequence of SEQ ID NO: 66. A truncation may be 1, 2, 3, 4, 5, 6, 7, 8, or 9 nucleotides shorter than the sequence of SEQ ID NO: 67. A truncation may be 1, 2, 3, 4, 5, 6, 7, 8, or 9 nucleotides shorter than the sequence of SEQ ID NO: 68. A truncation may be 1, 2, 3, 4, 5, 6, 7, 8, or 9 nucleotides shorter than the sequence of SEQ ID NO: 69. A truncation may be 1, 2, 3, 4, 5, 6, 7, 8, or 9 nucleotides shorter than the sequence of SEQ ID NO: 70. A truncation may be 1, 2, 3, 4, 5, 6, 7, 8, or 9 nucleotides shorter than the sequence of SEQ ID NO: 71. A truncation may be 1, 2, 3, 4, 5, 6, 7, 8, or 9 nucleotides shorter than the sequence of SEQ ID NO: 72. A truncation may be 1, 2, 3, 4, 5, 6, 7, 8, or 9 nucleotides shorter than the sequence of SEQ ID NO: 73. A truncation may be 1, 2, 3, 4, 5, 6, 7, 8, or 9 nucleotides shorter than the sequence of SEQ ID NO: 74. A truncation may be 1, 2, 3, 4, 5, 6, 7, 8, or 9 nucleotides shorter than the sequence of SEQ ID NO: 75. A truncation may be 1, 2, 3, 4, 5, 6, 7, 8, or 9 nucleotides shorter than the sequence of SEQ ID NO: 76. A truncation may be 1, 2, 3, 4, 5, 6, 7, 8, or 9 nucleotides shorter than the sequence of SEQ ID NO: 77. A truncation may be 1, 2, 3, 4, 5, 6, 7, 8, or 9 nucleotides shorter than the sequence of SEQ ID NO: 78. A truncation may be 1, 2, 3, 4, 5, 6, 7, 8, or 9 nucleotides shorter than any of the sequences of SEQ ID NOs: 79-102.

In some embodiments, the gRNA may be encoded by or bind and target and/or hybridize to a polynucleotide sequence comprising SEQ ID NO: 53 or SEQ ID NO: 54 or a complement thereof, or a variant thereof, or a truncation thereof. In some embodiments, the gRNA comprises a polynucleotide sequence selected from SEQ ID NO: 103 and SEQ ID NO: 104 or a complement thereof, or a variant thereof, or a truncation thereof.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

d. Donor Sequence

The CRISPR/Cas9-based gene editing system may include at least one donor sequence. A donor sequence comprises a polynucleotide sequence to be inserted into a genome. A donor sequence may comprise a wild-type sequence of a gene.

The gRNA and donor sequence may be present in a variety of molar ratios. The molar ratio between the gRNA and donor sequence may be 1:1, or 1:15, or from 5:1 to 1:10, or from 1:1 to 1:5. The molar ratio between the gRNA and donor sequence may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the gRNA and donor sequence may be less than 20:1, less than 15:1, less than 10:1, less than 9:1, less than 8:1, less than 7:1, less than 6:1, less than 5:1, less than 4:1, less than 3:1, less than 2:1, or less than 1:1.

e. Repair Pathways

The CRISPR/Cas9-based gene editing system may be used to introduce site-specific double strand breaks at targeted genomic loci, such as the dystrophin gene, the utrophin gene, or the dystrophin gene within the Xp21 locus. Site-specific double-strand breaks are created when the CRISPR/Cas9-based gene editing system binds to a target DNA sequence, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

i) Homology-Directed Repair (HDR)

Restoration of protein expression from a gene may involve homology-directed repair (HDR). A donor template may be administered to a cell. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

ii) NHEJ

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 6. Genetic Constructs

The CRISPR/Cas9-based gene editing system may be encoded by or comprised within a genetic construct. The genetic construct, such as a plasmid or expression vector, may comprise a nucleic acid that encodes the CRISPR/Cas9-based gene editing system and/or at least one of the gRNAs. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally two Cas9 molecules or fusion proteins, e.g. a first Cas9 molecule and a second Cas9 molecule. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein.

Genetic constructs may include polynucleotides such as vectors and plasmids. Genetic constructs may include transposons. For example, the genetic construct may be a transposon encoding a Cas9 molecule or fusion protein and/or at least one gRNA molecule. The transposon may be stably integrated into the genome of a subject. The transposon may be co-administered with a transposase or a polynucleotide encoding the transposase. Transposon systems known in the art may include, for example, piggybac or sleeping beauty systems. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The initiation and termination codon may be in frame with the CRISPR/Cas-based gene editing system coding sequence.

The genetic construct may also comprise a promoter that is operably linked to the CRISPR/Cas-based gene editing system coding sequence. In some embodiments, the promoter is operably linked to a polynucleotide encoding a gRNA and a Cas9 scaffold. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human 1-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”) or puromycin (“Puro”).

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species. In some embodiments, the viral vector is a lentiviral vector. The lentivirus is a genus belonging to the Retroviridae family that infects humans and other mammals.

AAV vectors or lentiviral vectors may be used to deliver CRISPR/Cas9-based gene editing systems using various construct configurations. For example, AAV vectors or lentiviral vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector or lentiviral vector. In some embodiments, the AAV vector has a 4.7 kb packaging limit. In some embodiments, the lentiviral vector has a 9.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

The genetic construct may comprise or encode a polynucleotide sequence selected from SEQ ID NOs: 55-107. The genetic construct may comprise a polynucleotide sequence of SEQ ID NO: 55. The genetic construct may comprise a polynucleotide sequence of SEQ ID NO: 56. The genetic construct may comprise a polynucleotide sequence of SEQ ID NO: 57. The genetic construct may comprise a polynucleotide sequence of SEQ ID NO: 58. The genetic construct may comprise a polynucleotide sequence of SEQ ID NO: 59. The genetic construct may comprise a polynucleotide sequence of SEQ ID NO: 60. The genetic construct may comprise a polynucleotide sequence of SEQ ID NO: 61. The genetic construct may comprise a polynucleotide sequence of SEQ ID NO: 62. The genetic construct may comprise a polynucleotide sequence of SEQ ID NO: 63. The genetic construct may comprise a polynucleotide sequence of SEQ ID NO: 64. The genetic construct may comprise a polynucleotide sequence of SEQ ID NO: 65. The genetic construct may comprise a polynucleotide sequence of SEQ ID NO: 66. The genetic construct may comprise a polynucleotide sequence of SEQ ID NO: 67. The genetic construct may comprise a polynucleotide sequence of SEQ ID NO: 68. The genetic construct may comprise a polynucleotide sequence of SEQ ID NO: 69. The genetic construct may comprise a polynucleotide sequence of SEQ ID NO: 70. The genetic construct may comprise a polynucleotide sequence of SEQ ID NO: 71. The genetic construct may comprise a polynucleotide sequence of SEQ ID NO: 72. The genetic construct may comprise a polynucleotide sequence of SEQ ID NO: 73. The genetic construct may comprise a polynucleotide sequence of SEQ ID NO: 74. The genetic construct may comprise a polynucleotide sequence of SEQ ID NO: 75. The genetic construct may comprise a polynucleotide sequence of SEQ ID NO: 76. The genetic construct may comprise a polynucleotide sequence of SEQ ID NO: 77. The genetic construct may comprise a polynucleotide sequence of SEQ ID NO: 78. The genetic construct may comprise a polynucleotide sequence of SEQ ID NO: 37. The genetic construct may comprise a polynucleotide sequence of SEQ ID NO: 53. The genetic construct may comprise a polynucleotide sequence of SEQ ID NO: 54.

### 7. Pharmaceutical Compositions

Further provided herein are pharmaceutical compositions comprising the above-described genetic constructs or gene editing systems. In some embodiments, the pharmaceutical composition may comprise about 1 ng to about 10 mg of DNA encoding the CRISPR/Cas-based gene editing system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 8. Administration

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector lIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein. In some embodiments, the cell is a cell type currently under investigation for cell-based therapies, including, but not limited to, immortalized myoblast cells, such as wild-type and DMD patient derived lines, primal DMD dermal fibroblasts, stem cells such as induced pluripotent stem cells, bone marrow-derived progenitors, skeletal muscle progenitors, human skeletal myoblasts from DMD patients, CD 133+ cells, mesoangioblasts, cardiomyocytes, hepatocytes, chondrocytes, mesenchymal progenitor cells, hematopoetic stem cells, muscle cells, satellite cells, smooth muscle cells, and MyoD- or Pax7-transduced cells, or other myogenic progenitor cells. Immortalization of human myogenic cells can be used for clonal derivation of genetically corrected myogenic cells. Cells can be modified ex vivo to isolate and expand clonal populations of immortalized DMD myoblasts that include a genetically corrected or restored dystrophin gene and are free of other nuclease-introduced mutations in protein coding regions of the genome. Cells can be modified in vitro to screen CRISPR/Cas-based gene editing systems, any cell line known to one of skill in the art may be used. In some embodiments, the cell line is human embryonic kidney 293 (HEK293) or HEK293T cells. In some embodiments, the virus is added to the cells at a multiplicity of infection (MOI) of at least 0.1, at least 0.2, at least 0.3, at least 0.4, at least 0.5, at least 0.6, at least 0.7, at least 0.8, at least 0.9, or at least 1.

### 9. Kits

Provided herein is a kit, which may be used to restore function to a dystrophin gene. The kit comprises genetic constructs or a composition comprising the same, and instructions for using said composition. In some embodiments, the kit comprises at least one gRNA comprising or encoded by a polynucleotide sequence selected from SEQ ID NOs: 5-78, a complement thereof, a variant thereof, or fragment thereof, or at least one gRNA that binds and targets a polynucleotide sequence comprising or selected from SEQ ID NOs: 55-78, a complement thereof, a variant thereof, or fragment thereof. In some embodiments, the kit comprises at least one gRNA comprising a polynucleotide sequence selected from SEQ ID NOs: 79-102, or a complement thereof, or a variant thereof, or a truncation thereof. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising thereof for restoring function to a dystrophin gene may include a modified AAV vector that includes a gRNA molecule(s) and a Cas9 protein or fusion protein, as described above, that specifically binds and cleaves a region of the dystrophin gene. The CRISPR/Cas-based gene editing system, as described above, may be included in the kit to specifically bind and target a particular region, for example, exon 51, in the gene.

### 10. Methods

a. Methods of Screening for Pairs of gRNA Molecules for Editing a Genomic Nucleic Acid

Provided herein are methods of high-throughput screening for pairs of gRNA molecules to be used in CRISPR/Cas9-based gene editing systems. The methods may include generating a plurality of pairs of gRNA molecules that target different nucleic acid sequences. For example, the methods of screening may include quantifying the level of editing a nucleic acid, for each individual pair of a plurality of gRNA molecules. The nucleic acid may be genomic nucleic acid. Each pair of gRNA molecules can include a first gRNA molecule that targets a first nucleic acid sequence and a second gRNA molecule that targets a second nucleic acid sequence. The first and second nucleic acid sequences can be a portion of different introns, the same intron, different exons, or the same exon. In some embodiments, the first nucleic acid sequence is a portion of intron 50 of the human dystrophin gene and the second nucleic acid sequence is a portion of intron 51 of the human dystrophin gene. The first nucleic acid sequence and the second nucleic acid sequence may each be at least 5 kb from an exon. The first nucleic acid sequence and the second nucleic acid sequence may each be about 1 kb from an exon, about 2 kb from an exon, about 3 kb from an exon, about 4 kb from an exon, about 5 kb from an exon, about 6 kb from an exon, about 7 kb from an exon, about 8 kb from an exon, about 9 kb from an exon, or about 10 kb from an exon. Each gRNA may comprise 0 consecutive thymine nucleotides (T's). Each gRNA may include at most 4 consecutive T's, at most 3 consecutive T's, or at most 2 consecutive T's. Each gRNA may have no predicted off-target binding in the human or mouse genome. Each gRNA can have at most 1 mismatch, 2 mismatches, 3 mismatches, 4 mismatches, or 5 mismatches with the nucleic acid sequence it targets. Each gRNA can be 95% complementary to the target nucleic acid sequence, 96% complementary to the target nucleic acid sequence, 97% complementary to the target nucleic acid sequence, 98% complementary to the target nucleic acid sequence, 99% complementary to the target nucleic acid sequence, or 100% complementary to the target nucleic acid sequence.

In some embodiments, the first nucleic acid sequence comprises a first intron of the dystrophin gene and the second nucleic acid sequence comprises a second intron of the dystrophin gene. In some embodiments, the first intron is adjacent to one side of the at least one exon and the second intron is adjacent to the other side of the at least one exon. In some embodiments, the at least one exon is in between the first and second introns in the genomic nucleic acid. In some embodiments, the genomic nucleic acid comprises two or more exons of a dystrophin gene, and the first intron is adjacent to one side of the two or more exons and the second intron is adjacent to the other side of the two or more exons. In some embodiments, the two or more exons are in between the first and second introns in the genomic nucleic acid.

The method may include expressing a Cas9 protein or a fusion protein comprising the Cas9 protein, and the plurality of pairs of gRNA molecules in a plurality of cells. One pair of gRNA molecules may be expressed in each cell. The first gRNA may direct the Cas9 protein or fusion protein to cut the first nucleic acid sequence and the second gRNA may direct the Cas9 protein or fusion protein to cut the second nucleic acid sequence, thereby forming an excised nucleic acid and a new junction in the genomic nucleic acid.

In some embodiments, the expression is effected by transfecting the plurality of cells with a plurality of vectors. Each cell may be transfected with a first vector encoding one pair of gRNA molecules and a second vector encoding the Cas9 protein or fusion protein. In some embodiments, each cell is transfected with a different first vector encoding a different pair of gRNA molecules. The method may include transfecting cells with lentiviruses comprising a vector encoding a Cas9 protein or a fusion protein comprising the Cas9 protein such that the cells express the Cas9 protein. The method may also include transfecting the Cas9 protein-expressing cells with a variety of lentiviruses, each lentivirus comprising a vector encoding a pair of gRNA molecules. Each virus comprises a different vector encoding a different pair of gRNA molecules. Each cell is transfected with a different lentivirus. In the cell, the first gRNA molecule directs the Cas9 protein to the first nucleic acid sequence and the second gRNA molecule directs the Cas9 protein to the second nucleic acid sequence. This introduces site-specific double strand breaks at targeted genomic loci and excision of a nucleic acid resulting in modification of the genomic nucleic acid. The method may further include isolation of the modified genomic nucleic acid (i.e., genomic DNA) from the cells by methods known in the art, such as a DNA extraction kit.

In some embodiments, the excised nucleic acid comprises exon 51. In some embodiments, the first nucleic acid sequence is within intron 50 of the dystrophin gene. In some embodiments, the second nucleic acid sequence is within intron 51 of the dystrophin gene.

The junctions in the isolated genomic nucleic acid may be enriched by first binding probes with specificity for a portion of the first nucleic acid sequence. The genomic nucleic acid bound to the probes can be isolated using any method known the in art, such as with magnetic beads, such as streptavidin coated beads. Next, the isolated genomic nucleic acid can be incubated with probes with specificity for a portion of the second nucleic acid sequence and isolated. The probes may bind at the site of the double strand breaks (i.e., the new junction) in the genomic nucleic acid. At least 1 probe may specifically bind the new junction, at least 2 probes may specifically bind the new junction, at least 3 probes may specifically bind the new junction, at least 4 probes may specifically bind the new junction, and at least 5 probes may specifically bind the new junction. The probes may each bind to the new junction and a different portion of the first nucleic acid sequence. In some embodiments, the genomic nucleic acid is contacted with a first pool of probes, wherein one or more different probes specifically bind to each new junction and a portion of the first nucleic acid sequence. In some embodiments, the genomic nucleic acid is contacted with a first pool of probes, wherein at least 3 different probes specifically bind to each new junction and a portion of the first nucleic acid sequence. The genomic nucleic acid bound to the first pool of probes may be isolated, and then the genomic nucleic acid bound to the first pool of probes may be contacted with a second pool of probes, wherein one or more different probes specifically bind to each new junction and a portion of the second nucleic acid sequence. The genomic nucleic acid bound to the first pool of probes may be isolated, and then the genomic nucleic acid bound to the first pool of probes may be contacted with a second pool of probes, wherein at least 3 different probes specifically bind to each new junction and a portion of the second nucleic acid sequence. The genomic nucleic acid bound to the second pool of probes may be isolated.

The probes may bind 10 bp away from the site of the double strand break in the genomic nucleic acid. The probes may bind 20 bp away from the site of the double strand break in the genomic nucleic acid. The probes may bind 30 bp away from the site of the double strand break in the genomic nucleic acid. The probes may have a length of about 100 bp to about 140 bp, about 105 bp to about 135 bp, about 110 bp to about 130 bp, or about 115 bp to about 125 bp. The probes may have a length of about 102 bp, about 103 bp, about 104 bp, about 105 bp, about 106 bp, about 107 bp, about 108 bp, about 109 bp, about 110 bp, about 111 bp, about 112 bp, about 113 bp, about 114 bp, about 115 bp, about 116 bp, about 117 bp, about 118 bp, about 119 bp, about 120 bp, about 121 bp, about 122 bp, about 123 bp, about 124 bp, about 125 bp, about 126 bp, about 127 bp, about 128 bp, about 129 bp, about 130 bp, about 131 bp, about 132 bp, about 133 bp, about 134 bp, about 135 bp, about 136 bp, about 137 bp, about 138 bp, about 139 bp, or about 140 bp. In some embodiments, the probes have a length of about 120 bp.

Each probe may comprise any suitable affinity label known in the art. In some embodiments, the probes are biotinylated probes.

b. Methods of Identifying a Pair of gRNA Molecules

Provided herein are methods of identifying pairs of gRNA molecules that produce unique junctions in the genomic nucleic acid at a relatively high frequency and high deletion efficiency. In addition to the methods as described above, the methods may include sequencing the genomic nucleic acid that bound to the probes with specificity for a portion of the first nucleic acid sequence and the probes with specificity for a portion of the second nucleic acid sequence. Sequencing may be performed by methods known in the art, such as using an Illumina NextSeq. Further, the method can include aligning the sequenced genomic nucleic acid to identify the new junctions made by the CRISPR/Cas9-based gene editing system comprising the pairs of gRNA molecules and assigning each new junction to the corresponding pair of gRNA molecules. The pairs of gRNA molecules with the greatest number of new junctions with the greatest deletion efficiency can be determined from the preceding assignment, where the deletion efficiency can be measured by the frequency of each new junction.

Further provided herein is a pair of gRNA molecules identified by the method detailed herein. Further provided herein is a CRISPR/Cas9 system comprising such a pair of gRNA molecules. The gRNA may by encoded by or bind and target a polynucleotide sequence comprising at least one of SEQ ID NOs: 55-78. The gRNA may comprise a polynucleotide sequence selected from SEQ ID NOs: 79-102.

c. Methods of Editing a Genomic Nucleic Acid in a Subject

Provided herein are methods of editing a genomic nucleic acid in a cell or subject. The genomic nucleic acid may be a mutant dystrophin gene or a mutant human dystrophin gene that causes disease, such as DMD. The method can include administering to a cell or a subject a presently disclosed system or genetic construct or a composition comprising thereof as described above. The method can comprise administering to the skeletal muscle and/or cardiac muscle of the subject the presently disclosed system or genetic construct or a composition comprising the same for editing a genomic nucleic acid in skeletal muscle and/or cardiac muscle, as described above. Use of the presently disclosed system or genetic construct or a composition comprising the same to deliver the CRISPR/Cas9-based gene editing system to the skeletal muscle or cardiac muscle may restore the expression of a fully-functional or partially functional protein. The CRISPR/Cas9-based gene editing system may be used to introduce site-specific double strand breaks at targeted genomic loci. Site-specific double-strand breaks are created when the CRISPR/Cas9-based gene editing system binds to target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway. The method may include administering a CRISPR/Cas9-based gene editing system, such as administering a Cas9 protein or Cas9 fusion protein, a nucleotide sequence encoding said Cas9 protein or Cas9 fusion protein, and/or at least one gRNA, wherein the gRNAs target different DNA sequences. The target DNA sequences may be overlapping. The number of gRNA administered to the cell may be at least 1 gRNA, at least 2 different gRNA, at least 3 different gRNA at least 4 different gRNA, at least 5 different gRNA, at least 6 different gRNA, at least 7 different gRNA, at least 8 different gRNA, at least 9 different gRNA, at least 10 different gRNA, at least 15 different gRNA, at least 20 different gRNA, at least 30 different gRNA, or at least 50 different gRNA, as described above. This strategy may integrate the rapid and robust assembly of active CRISPR/Cas9-based gene editing system with an efficient gene editing method for the treatment of genetic diseases caused by mutations in nonessential coding regions that cause frameshifts, premature stop codons, aberrant splice donor sites or aberrant splice acceptor sites.

d. Methods of Correcting a Mutant Gene and Treating a Subject

Provided herein are methods of correcting a mutant gene (for example, a mutant dystrophin gene) in a cell and treating a subject suffering from a genetic disease, such as DMD. The method can include administering to a cell or a subject a presently disclosed system or genetic construct or a composition comprising the same as described above. The method can comprise administering to the skeletal muscle and/or cardiac muscle of the subject the presently disclosed system or genetic construct or a composition comprising the same for genome editing in skeletal muscle and/or cardiac muscle, as described above. Use of the presently disclosed system or genetic construct or a composition comprising the same to deliver the CRISPR/Cas9-based gene editing system to the skeletal muscle or cardiac muscle may restore the expression of a fully-functional or partially functional protein with a repair template or donor DNA, which can replace the entire gene or the region containing the mutation. The CRISPR/Cas9-based gene editing system may be used to introduce site-specific double strand breaks at targeted genomic loci. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

The present disclosure is further directed to genome editing with a CRISPR/Cas9-based gene editing system without a repair template, which can efficiently correct the reading frame and restore the expression of a functional protein involved in a genetic disease. The disclosed CRISPR/Cas9-based gene editing system and methods may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active CRISPR/Cas9-based gene editing system with an efficient gene editing method for the treatment of genetic diseases caused by mutations in nonessential coding regions that cause frameshifts, premature stop codons, aberrant splice donor sites or aberrant splice acceptor sites.

The present disclosure also provides methods of correcting a mutant gene in a cell and treating a subject suffering from a genetic disease, such as DMD. The method may include administering to a cell or subject a CRISPR/Cas9-based gene editing system, a polynucleotide or vector encoding said CRISPR/Cas9-based gene editing system, or a composition of said CRISPR/Cas9-based gene editing system as described above. The method may include administering a CRISPR/Cas9-based gene editing system, such as administering a Cas9 protein or Cas9 fusion protein containing a second domain having nuclease activity, a nucleotide sequence encoding said Cas9 protein or Cas9 fusion protein, and/or at least one gRNA. The gRNAs may target different DNA sequences.

e. Methods of Treating Disease

Provided herein are methods of treating a subject in need thereof. The method may comprise administering to a tissue of a subject the presently disclosed system or genetic construct or a composition comprising thereof, as described above. In certain embodiments, the method may comprise administering to the skeletal muscle or cardiac muscle of the subject the presently disclosed system or genetic construct or composition comprising thereof, as described above. In certain embodiments, the method may comprise administering to a vein of the subject the presently disclosed system or genetic construct or composition comprising thereof, as described above. In certain embodiments, the subject is suffering from a skeletal muscle or cardiac muscle condition causing degeneration or weakness or a genetic disease. For example, the subject may be suffering from Duchenne muscular dystrophy, as described above.

i) Duchenne Muscular Dystrophy

The method, as described above, may be used for correcting the dystrophin gene and recovering full-functional or partially-functional protein expression of said mutated dystrophin gene. In some aspects and embodiments the disclosure provides a method for reducing the effects (for example, clinical symptoms or indications) of DMD in a subject. In some aspects and embodiments the disclosure provides a method for treating DMD in a subject. In some aspects and embodiments the disclosure provides a method for preventing DMD in a subject. In some aspects and embodiments the disclosure provides a method for preventing further progression of DMD in a subject.

f. Methods of Screening Therapeutic Agents

Provided herein are methods of screening therapeutic agents for treating Duchenne muscular dystrophy (DMD). The method may include administering one or more therapeutic agents to the transgenic mouse detailed herein. The one or more therapeutic agents may be a small molecule, anti-sense RNA, vector, CRISPR/Cas gene editing system, or biological agent, or a combination thereof. The vector may be a viral vector encoding a gene of interest, such as an AAV vector. In some embodiments, the mouse after administration of the one or more therapeutic agents exhibits increased lifespan, reduced body mass, increased body strength, increased motor coordination, increased balance, increased forelimb strength, reduced muscle injury, and/or reduced CK level compared to before administration of the one or more therapeutic agents. In some embodiments, the mouse after administration of the one or more therapeutic agents exhibits increased expression of a dystrophin gene as compared to before administration of the one or more therapeutic agents. The dystrophin gene may be a truncated human dystrophin gene. The truncated human dystrophin gene may include a plurality of deletions relative to a wild-type human dystrophin gene. In some embodiments, at least one of the deletions is in exon 52.

### 11. Examples

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Screening a Pool of gRNA Pairs for Exon Deletion

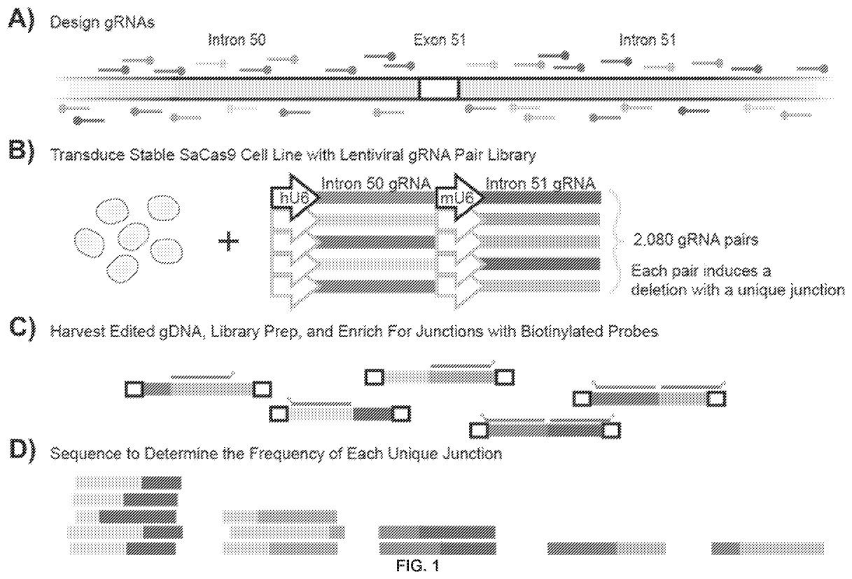

For SaCas9 deletion of human DMD exon 51, gRNAs within 5 kb of the exon with no poly T's and no predicted off-targets in the human genome with up to 3 mismatches were used (FIG. 1A). Individual gRNAs located in each relevant intron were identified computationally with GT scan software. Sequences with 4 or more consecutive T's were discarded as they would interfere with transcription. Potential binding sites to other locations in the genome were calculated, and only gRNAs with no predicted off-targets with up to 3 mismatched base pairs were selected. The library of gRNA pairs was created by pairing every gRNA in the first intron with every gRNA in the second intron. This resulted in 52 gRNAs in intron 50 and 40 gRNAs in intron 51, totaling 2,080 gRNA pairs (FIG. 1B). The sequence of each gRNA pair was synthesized as a single-stranded DNA oligo in a pooled format. This pool was amplified and cloned into a plasmid backbone between a human U6 promoter and an SaCas9 scaffold. This plasmid also had a puromycan selection cassette. After preparation of this plasmid pool, a second digestion between the two gRNAs was performed, and a second promoter (mouse U6) and a second SaCas9 scaffold was inserted into the plasmid. The result was a lentiviral plasmid pool where each plasmid contained hU6− intron 1 gRNA− SaCas9 scaffold− mU6− intron 2 gRNA− SaCas9 scaffold. This plasmid pool was then sequenced to confirm full coverage of the gRNA pair library, and then lentivirus was generated and subsequently titered. Each lentivirus contained one gRNA pair.

Lentivirus that expressed SaCas9 from a constitutive EFS promoter with a 2A hygromycin selection cassette was produced. HEK293T cells were transduced with the lentivirus, underwent hygromycin selection, and were then sorted into a 96-well plate with a single cell in each well. Clones of these single cells were grown up and stained for high expression of SaCas9. The Cas9-expressing HEK293T cells were transduced with the lentiviral library containing the gRNA pairs. The virus was added at an MOI of 0.2, such that at any cell received at most one viral particle, and thus one pair of gRNAs. The library was transduced at 1,000x coverage, such that each gRNA pair should have been introduced to approximately 1,000 cells. Cells were selected with puromycin for 5 days so that all non-transduced cells died. All of the cells were harvested 7 days after the initial transduction. Cells from the same line not treated with the library were also harvested.

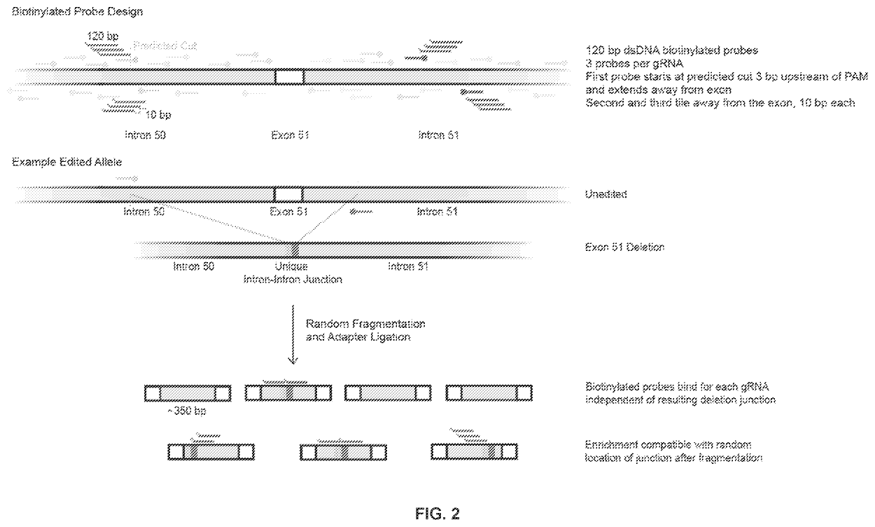

After treating a cell population with the library of gRNA pairs, junctions were enriched (FIG. 1C). Genomic DNA (gDNA) was extracted from the harvested cells with the Qiagen Blood and Cell Culture Midi Kit and DNA concentration was quantified. Using the Kapa HyperPlus Library Prep Kit, the gDNA was randomly fragmented and adapters were added to create ˜350 bp libraries from both the screen and non-treated gDNA. 20 ug of gDNA per sample was used to generate these libraries, maintaining 1000x coverage of the gRNA pair library. Two pools of dsDNA biotinylated probes were designed, one in each intron containing gRNAs (FIG. 2). Probes were designed for each gRNA and can theoretically bind independently of how the DNA has been edited, whether wild-type or any gRNA-to-gRNA exon deletion. They can also be applied to untreated DNA to determine the bias caused by the probes for sequencing some regions more than others. The probes were designed such that they began at the expected cut site of each gRNA, and extended away from the direction of the expected deletion 120 bp. Three of these 120 bp probes were designed for each gRNA, tiling back 10 bp away from the deletion each time. In order to enrich for edited fragments in the region of interest before sequencing, the sequencing libraries in step 5 were hybridized to the first intron probe pool, pulled down with streptavidin-coated beads, and then were subjected to a second hybridization and pulldown with the probe pool for the second intron. Only molecules with sequences targeted by both probe pools should have remained, thus enriching for molecules encoding an edited sequence. The libraries from the non-treated samples were also subjected to probe pulldown, but with both pools simultaneously. These samples were used to determine the sequencing coverage after pulldown to account for any bias the probes had to make certain regions more or less represented when sequenced. Another important normalization step was to account for the initial abundance of each gRNA pair in the starting lentiviral library. To measure this, PCR was performed on the gDNA harvested from library-treated cells at 500x coverage to amplify out the dual-gRNA lentiviral genome integrated into those cell's genomes.

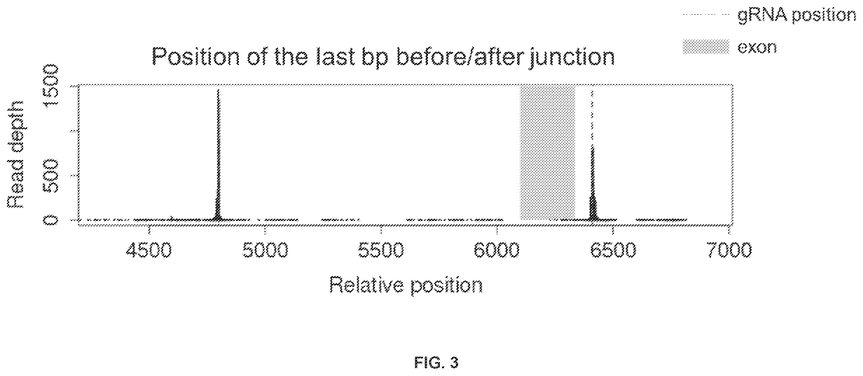

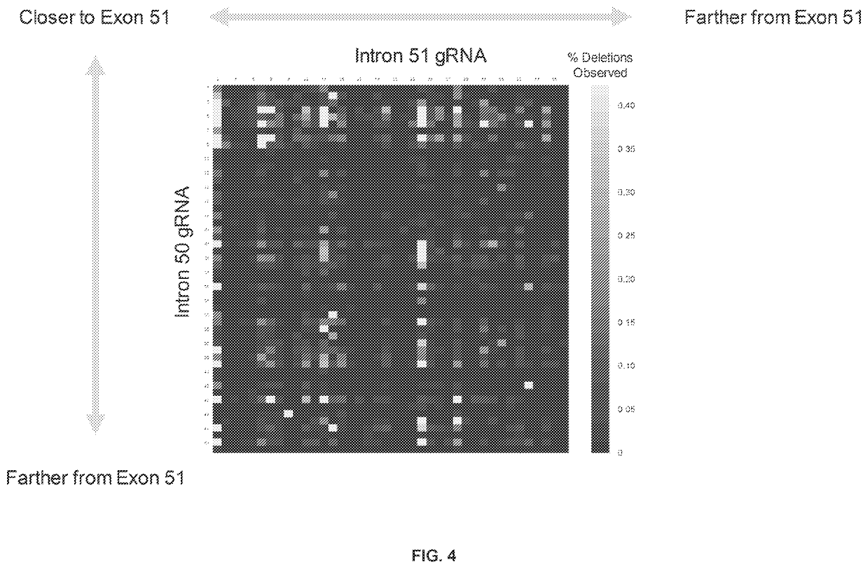

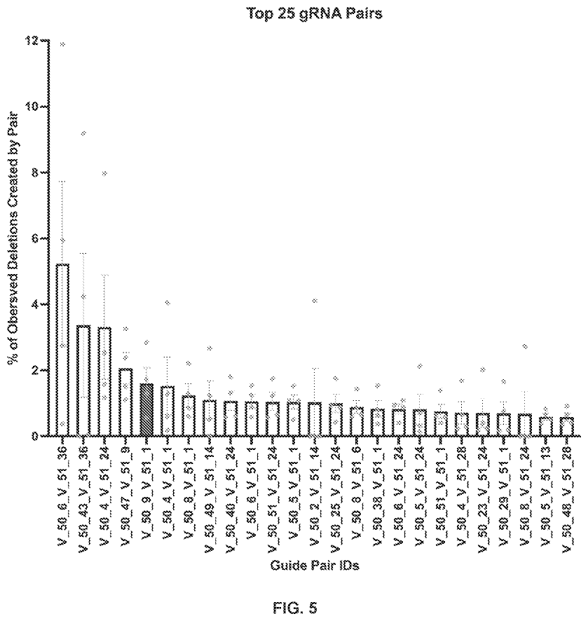

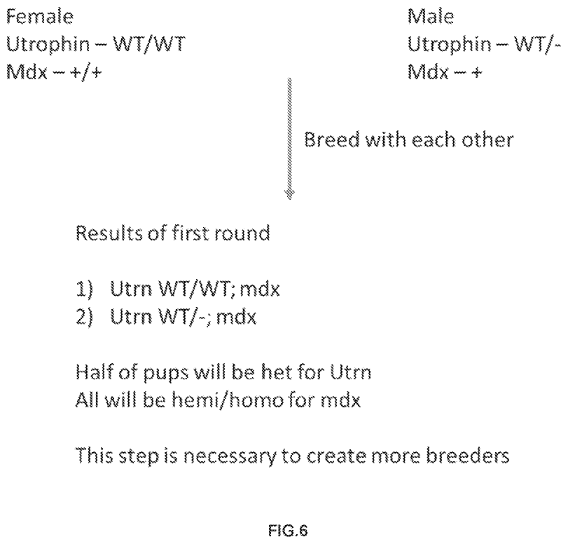

Despite the probe hybridization step used to enrich for edited sequences, the percent of sequencing reads containing a unique junction of one gRNA to another is low, around 0.065%. As a result, hundreds of millions of sequencing reads are necessary to achieve 100x coverage of the original gRNA pair library. All probe-hybridized samples were sequenced on an Illumina NextSeq, while the amplified gRNA sequences were sequenced on an Illumina MiSeq. The final step was to align the sequencing data to identify the novel junctions formed by exon deletions, assign each junction to the gRNA pair that created it, normalize the appropriate variables, and determine the relative efficiency of each gRNA pair (FIG. 1D). Since each gRNA pair yields a unique junction, the frequency of each junction was a direct measure of the deletion efficiency for a gRNA pair. The enrichment and sequencing methods were first tested on cells that only received a single gRNA pair (FIG. 3). This confirmed both the ability to detect the unique intron-intron junction as well as the hypothesis that the majority of deletions are the perfect ligation of the expected gRNA cut-sites. The frequency with which deletion-making gRNA pairs were identified by sequencing was normalized by initial gRNA abundance and bias introduced by probe hybridization. For all 2,080 pairs shown, many were not detected, but several pairs were detected with high frequency as measured by sequencing read counts for each gRNA pair (FIG. 4). The top 25 pairs identified with high frequency are shown in FIG. 5 and TABLE 1, including SEQ ID NOs: 55-78. The sequences in TABLE 1 are the sequences of the DNA target that the gRNA binds and targets. Corresponding RNA sequences are in TABLE 2.

TABLE 1

| Pair | Intron 50 | | | Intron 51 | | |
|---|---|---|---|---|---|---|
| Rank | gRNA ID | SEQ ID NO | Intron 50 gRNA 5′-3′ | gRNA ID | SEQ ID NO | Intron 51 gRNA 5′-3′ |
| 1 | V_50_6 | SEQ ID NO: 55 | CCCCAAAATGTGAAATACTTG | V_51_36 | SEQ ID NO: 71 | GTGGATAGCTTGATGGATAAT |
| 2 | V_50_4 | SEQ ID NO: 56 | ATAGCTGATAGTAGTTGAGAG | V_51_36 | SEQ ID NO: 71 | GTGGATAGCTTGATGGATAAT |
| 3 | V_50_4 | SEQ ID NO: 57 | TATGAATAGTGTTGGACACTG | V_51_24 | SEQ ID NO: 72 | AGGTAGATAGAGAGGCACAGT |
| 4 | V_50_4 | SEQ ID NO: 58 | GCAGGGTCGTATTGTTGTGCC | V_51_9 | SEQ ID NO: 73 | CAATTTGCCTTCTCCAATGAT |
| 5 | V_50_9 | SEQ ID NO: 59 | CACACAGCTGGGTTATCAGAG | V_51_1 | SEQ ID NO: 74 | AACTGGTGGGAAATGGTCTAG |
| 6 | V_50_4 | SEQ ID NO: 57 | TATGAATAGTGTTGGACACTG | V_51_1 | SEQ ID NO: 74 | AACTGGTGGGAAATGGTCTAG |
| 7 | V_50_8 | SEQ ID NO: 60 | ATACTAAATACCAACACACAG | V_51_1 | SEQ ID NO: 74 | AACTGGTGGGAAATGGTCTAG |
| 8 | V_50_4 | SEQ ID NO: 61 | ATAATCTAGAACGAACGGGTA | V_51_14 | SEQ ID NO: 75 | GTGTTATTACTTGCTACTGCA |
| 9 | V_50_4 | SEQ ID NO: 62 | TGAAATGGCCTGTGCTCATGA | V_51_24 | SEQ ID NO: 72 | AGGTAGATAGAGAGGCACAGT |
| 10 | V_50_6 | SEQ ID NO: 55 | CCCCAAAATGTGAAATACTTG | V_51_1 | SEQ ID NO: 74 | AACTGGTGGGAAATGGTCTAG |
| 11 | V_50_5 | SEQ ID NO: 63 | TAGAACGAACGGGTAAAGAGT | V_51_24 | SEQ ID NO: 74 | AACTGGTGGGAAATGGTCTAG |
| 12 | V_50_5 | SEQ ID NO: 64 | GTGTATTGCTTGTACTACTCA | V_51_1 | SEQ ID NO: 74 | AACTGGTGGGAAATGGTCTAG |
| 13 | V_50_2 | SEQ ID NO: 65 | AAGATATATAATGTCATGAAT | V_51_14 | SEQ ID NO: 75 | GTGTTATTACTTGCTACTGCA |
| 14 | V_50_2 | SEQ ID NO: 66 | TCTTGCATCTTGCACATGTCC | V_51_24 | SEQ ID NO: 72 | AGGTAGATAGAGAGGCACAGT |
| 15 | V_50_8 | SEQ ID NO: 60 | ATACTAAATACCAACACACAG | V_51_6 | SEQ ID NO: 76 | CAGAATCAAATATAATAGTCT |
| 16 | V_50_3 | SEQ ID NO: 67 | GAATGGAGAGAGGTAAGTCTG | V_51_1 | SEQ ID NO: 74 | AACTGGTGGGAAATGGTCTAG |
| 17 | V_50_6 | SEQ ID NO: 55 | CCCCAAAATGTGAAATACTTG | V_51_24 | SEQ ID NO: 72 | AGGTAGATAGAGAGGCACAGT |
| 18 | V_50_5 | SEQ ID NO: 64 | GTGTATTGCTTGTACTACTCA | V_51_24 | SEQ ID NO: 72 | AGGTAGATAGAGAGGCACAGT |
| 19 | V_50_5 | SEQ ID NO: 63 | TAGAACGAACGGGTAAAGAGT | V_51_1 | SEQ ID NO: 74 | AACTGGTGGGAAATGGTCTAG |
| 20 | V_50_4 | SEQ ID NO: 57 | TATGAATAGTGTTGGACACTG | V_51_28 | SEQ ID NO: 77 | GAATCCTTTGTTGCCAGACTG |
| 21 | V_50_2 | SEQ ID NO: 68 | TTATCTGCCCATGACTGGCGC | V_51_24 | SEQ ID NO: 72 | AGGTAGATAGAGAGGCACAGT |
| 22 | V_50_29 | SEQ ID NO: 69 | TTTCATTGTCTGCTCCAAGCA | V_51_1 | SEQ ID NO: 74 | AACTGGTGGGAAATGGTCTAG |
| 23 | V_50_8 | SEQ ID NO: 60 | ATACTAAATACCAACACACAG | V_51_24 | SEQ ID NO: 72 | AGGTAGATAGAGAGGCACAGT |
| 24 | V_50_5 | SEQ ID NO: 64 | GTGTATTGCTTGTACTACTCA | V_51_13 | SEQ ID NO: 78 | AGCAAGTAATAACACAAGCTT |
| 25 | V_50_4 | SEQ ID NO: 70 | TGAATCCATAATCTAGAACGA | V_51_28 | SEQ ID NO: 77 | GAATCCTTTGTTGCCAGACTG |

TABLE 2

| Pair | Intron 50 | | | Intron 51 | | |
|---|---|---|---|---|---|---|
| Rank | gRNA ID | SEQ ID NO | Intron 50 gRNA 5′-3′ | gRNA ID | SEQ ID NO | Intron 51 gRNA 5′-3′ |
| 1 | V_50_6 | SEQ ID NO: 79 | CCCCAAAAUGUGAAAUACUUG | V_51_36 | SEQ ID NO: 95 | GUGGAUAGCUUGAUGGAUAAU |
| 2 | V_50_4 | SEQ ID NO: 80 | AUAGCUGAUAGUAGUUGAGAG | V_51_36 | SEQ ID NO: 95 | GUGGAUAGCUUGAUGGAUAAU |
| 3 | V_50_4 | SEQ ID NO: 81 | UAUGAAUAGUGUUGGACACUG | V_51_24 | SEQ ID NO: 96 | AGGUAGAUAGAGAGGCACAGU |
| 4 | V_50_4 | SEQ ID NO: 82 | GCAGGGUCGUAUUGUUGUGCC | V_51_9 | SEQ ID NO: 97 | CAAUUUGCCUUCUCCAAUGAU |
| 5 | V_50_9 | SEQ ID NO: 83 | CACACAGCUGGGUUAUCAGAG | V_51_1 | SEQ ID NO: 98 | AACUGGUGGGAAAUGGUCUAG |
| 6 | V_50_4 | SEQ ID NO: 81 | UAUGAAUAGUGUUGGACACUG | V_51_1 | SEQ ID NO: 98 | AACUGGUGGGAAAUGGUCUAG |
| 7 | V_50_8 | SEQ ID NO: 84 | AUACUAAAUACCAACACACAG | V_51_1 | SEQ ID NO: 98 | AACUGGUGGGAAAUGGUCUAG |
| 8 | V_50_4 | SEQ ID NO: 85 | AUAAUCUAGAACGAACGGGUA | V_51_14 | SEQ ID NO: 99 | GUGUUAUUACUUGCUACUGCA |
| 9 | V_50_4 | SEQ ID NO: 86 | UGAAAUGGCCUGUGCUCAUGA | V_51_24 | SEQ ID NO: 96 | AGGUAGAUAGAGAGGCACAGU |
| 10 | V_50_6 | SEQ ID NO: 79 | CCCCAAAAUGUGAAAUACUUG | V_51_1 | SEQ ID NO: 98 | AACUGGUGGGAAAUGGUCUAG |
| 11 | V_50_5 | SEQ ID NO: 87 | UAGAACGAACGGGUAAAGAGU | V_51_24 | SEQ ID NO: 96 | AGGUAGAUAGAGAGGCACAGU |
| 12 | V_50_5 | SEQ ID NO: 88 | GUGUAUUGCUUGUACUACUCA | V_51_1 | SEQ ID NO: 98 | AACUGGUGGGAAAUGGUCUAG |
| 13 | V_50_2 | SEQ ID NO: 89 | AAGAUAUAUAAUGUCAUGAAU | V_51_14 | SEQ ID NO: 99 | GUGUUAUUACUUGCUACUGCA |
| 14 | V_50_2 | SEQ ID NO: 90 | UCUUGCAUCUUGCACAUGUCC | V_51_24 | SEQ ID NO: 96 | AGGUAGAUAGAGAGGCACAGU |
| 15 | V_50_8 | SEQ ID NO: 84 | AUACUAAAUACCAACACACAG | V_51_6 | SEQ ID NO: 100 | CAGAAUCAAAUAUAAUAGUCU |
| 16 | V_50_3 | SEQ ID NO: 91 | GAAUGGAGAGAGGUAAGUCUG | V_51_1 | SEQ ID NO: 98 | AACUGGUGGGAAAUGGUCUAG |
| 17 | V_50_6 | SEQ ID NO: 79 | CCCCAAAAUGUGAAAUACUUG | V_51_24 | SEQ ID NO: 96 | AGGUAGAUAGAGAGGCACAGU |
| 18 | V_50_5 | SEQ ID NO: 88 | GUGUAUUGCUUGUACUACUCA | V_51_24 | SEQ ID NO: 96 | AGGUAGAUAGAGAGGCACAGU |
| 19 | V_50_5 | SEQ ID NO: 87 | UAGAACGAACGGGUAAAGAGU | V_51_1 | SEQ ID NO: 98 | AACUGGUGGGAAAUGGUCUAG |
| 20 | V_50_4 | SEQ ID NO: 81 | UAUGAAUAGUGUUGGACACUG | V_51_28 | SEQ ID NO: 101 | GAAUCCUUUGUUGCCAGACUG |
| 21 | V_50_2 | SEQ ID NO: 92 | UUAUCUGCCCAUGACUGGCGC | V_51_24 | SEQ ID NO: 96 | AGGUAGAUAGAGAGGCACAGU |
| 22 | V_50_2 | SEQ ID NO: 93 | UUUCAUUGUCUGCUCCAAGCA | V_51_1 | SEQ ID NO: 98 | AACUGGUGGGAAAUGGUCUAG |
| 23 | V_50_8 | SEQ ID NO: 84 | AUACUAAAUACCAACACACAG | V_51_24 | SEQ ID NO: 96 | AGGUAGAUAGAGAGGCACAGU |
| 24 | V_50_5 | SEQ ID NO: 88 | GUGUAUUGCUUGUACUACUCA | V_51_13 | SEQ ID NO: 102 | AGCAAGUAAUAACACAAGCUU |
| 25 | V_50_4 | SEQ ID NO: 94 | UGAAUCCAUAAUCUAGAACGA | V_51_28 | SEQ ID NO: 101 | GAAUCCUUUGUUGCCAGACUG |

### Example 2

### Generation of Dystrophin and Utrophin Double Knockout Mouse

One humanized mouse model of DMD is based on the mdx mouse model described by C. E. Nelson et al., Science 10.1126/science.aad5143 (2015). The mdx mouse carries a nonsense mutation in exon 23 of the mouse dystrophin gene, which results in production of a full-length dystrophin mRNA transcript and encodes a truncated dystrophin protein. These molecular changes are accompanied by functional changes including reduced twitch and tetanic force in mdx muscle. The mdx mouse has been humanized by the addition of a full-length human dystrophin transgene comprising a deletion of exon 52 (“hDMDΔ52/mdx mouse”).

The hDMDΔ52/mdx mice were made by injecting a CRISPR/Cas9 system including a S. pyogenes Cas9 molecule and a pair of gRNAs targeting intron 51 and intron 52 of the human dystrophin gene, respectively, to the embryos of mdx mice containing the human dystrophin transgene. No dystrophin protein is detected in the heart and tibialis anterior muscle of the hDMDΔ52/mdx mice.

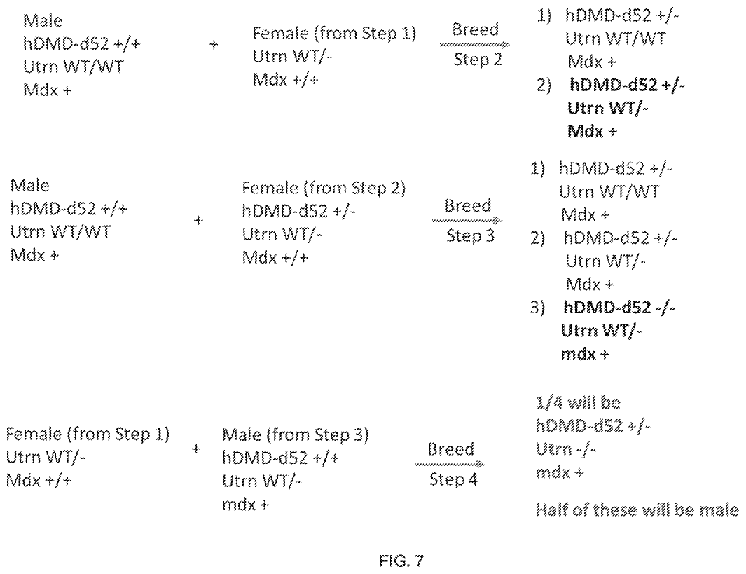

The + indicates the mice have 1 allele for the mutation or gene. For example, hDMDΔ52+/+ indicates that the mice have two positive alleles (i.e. homozygous) for the hDMDΔ52 mutation. These mice were used for breeding purposes and are dystrophin null. For the following studies, hDMDΔ52+/−; mdx mice (i.e. dystrophin null) were used. Male breeders (Utrn−/−; mdx) were purchased from the Jackson laboratory (stock #014563) and bred to mdx homozygous females to obtain Utrn+/−; mdx females (FIG. 6). Utrn+/−; mdx females were bred with male hDMDΔ52+/+; mdx mice to obtain hDMDΔ52+/−; Utrn+/−; mdx females (FIG. 7, step 2). Male hDMDΔ52+/+; mdx mice were bred with hDMDΔ52+/−; Utrn+/−; mdx females to obtain hDMDΔ52+/+; Utrn+/−; mdx males (FIG. 7, step 3). Male hDMDΔ52+/+; Utrn+/−; mdx were bred with Utrn+/−; mdx females to obtain hDMDΔ52+/−; Utrn−/−; mdx male study mice (FIG. 7, step 4).

### Example 3

### Loss of Utrophin Exacerbates the DMD Phenotype of hDMDΔ52/Mdx Mice

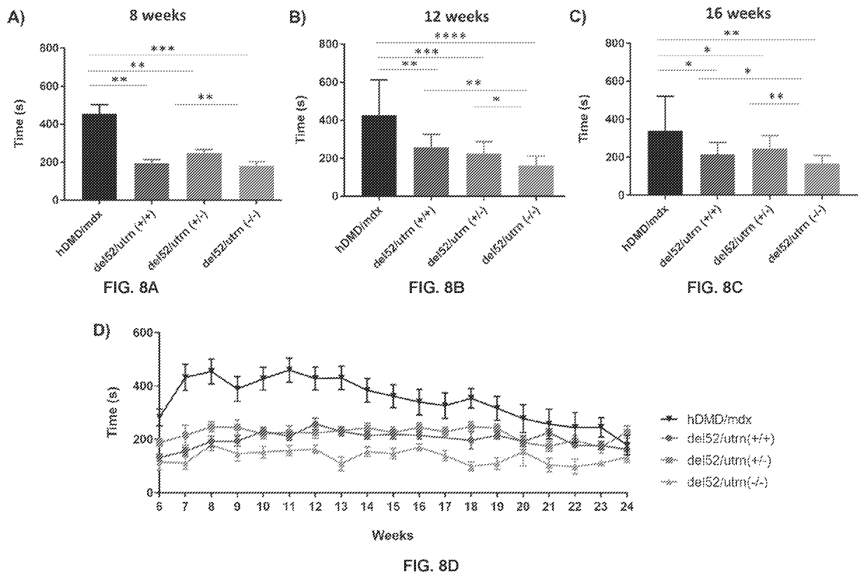

Mice were subjected to rotarod testing beginning at 6 weeks of age to assess motor coordination, whole body strength, and balance. Mice were placed on the rotarod, which accelerated from 4 to 40 rpm over a period of 5 min. The time to first fall was recorded. If mice fell within 30 seconds of the run, they were placed back on the rotarod for a second attempt. Data from isolated time points (8, 12, and 16 weeks) are shown in FIG. 8A-FIG. 8C. hDMDΔ52/mdx/Utrn KO mice (i.e. hDMDΔ52+/−; Utrn−/−; mdx) displayed significantly shortened running times compared to Utrn WT (i.e. hDMDΔ52+/−; Utrn+/+; mdx) and Utrn het (i.e. hDMDΔ52+/−; Utrn+/−; mdx) mice at later time points. Rotarod performance over time is shown in FIG. 8D, demonstrating significantly decreased running times for Utrn KO mice. Statistical analysis was performed using a t-test with Welch's correction to compare groups in FIG. 8A-FIG. 8C. A two-way ANOVA with Tukey's test was performed for rotarod performance over time (TABLE 3). Overall, these data demonstrate that loss of utrophin exacerbates the phenotype of the hDMDΔ52/mdx mice.

TABLE 3

| Comparison | P value |
|---|---|
| hDMD vs. del52/utrn WT | <0.00001 |
| hDMD vs. del52/utrn het | <0.00001 |
| hDMD vs. del52/utrn KO | <0.00001 |
| del52/utrn WT vs. del52/utrn het | 0.1778 (n.s.) |
| del52/utrn WT vs. del52/utrn KO | <0.00001 |
| del52/utrn het vs. del52/utrn KO | <0.00001 |

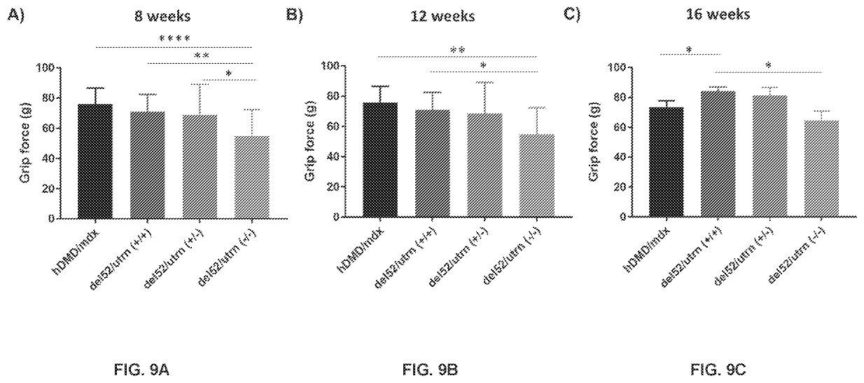

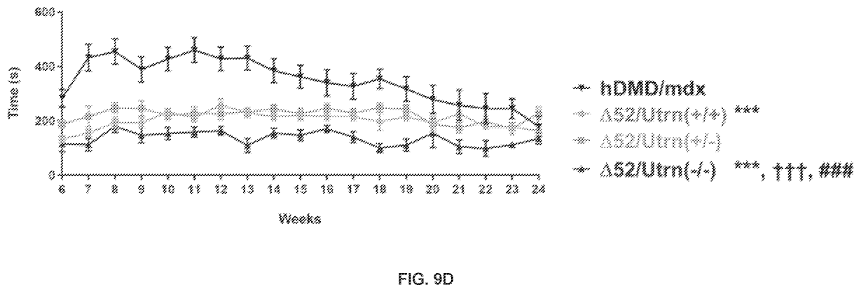

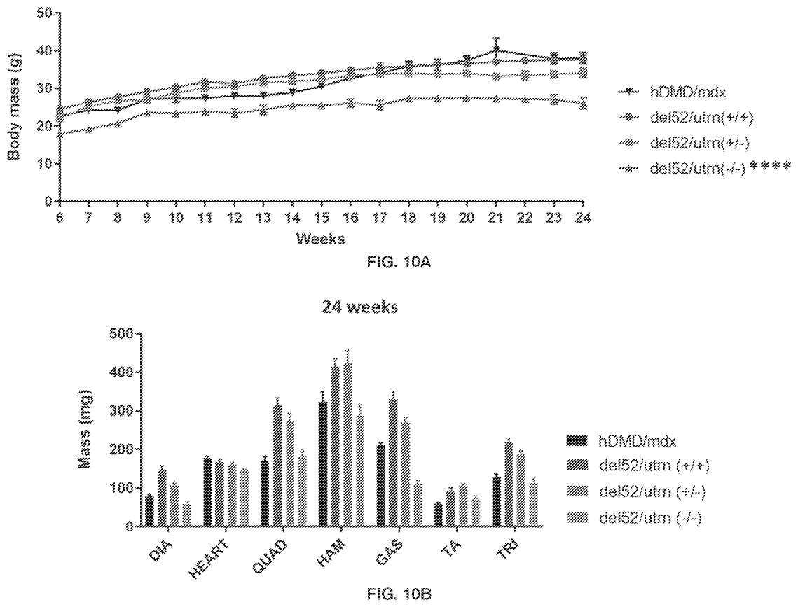

Mice were subjected to forelimb grip strength testing to assess forelimb strength. Data from isolated time points are shown in FIG. 9A, FIG. 9B, and FIG. 9C. hDMDΔ52/mdx/Utrn KO mice displayed decreased grip strength compared to Utrn WT and Utrn het mice, particularly at 8 weeks of age. Statistical analysis was performed using a t-test with Welch's correction to compare groups. Results from a rotarod assay are shown in FIG. 9D. These data indicate that loss of utrophin exacerbates the phenotype of the hDMDΔ52/mdx mice.

### Example 4

### Loss of Utrophin Exacerbates the DMD Physiological Characteristics of hDMDΔ52/Mdx Mice

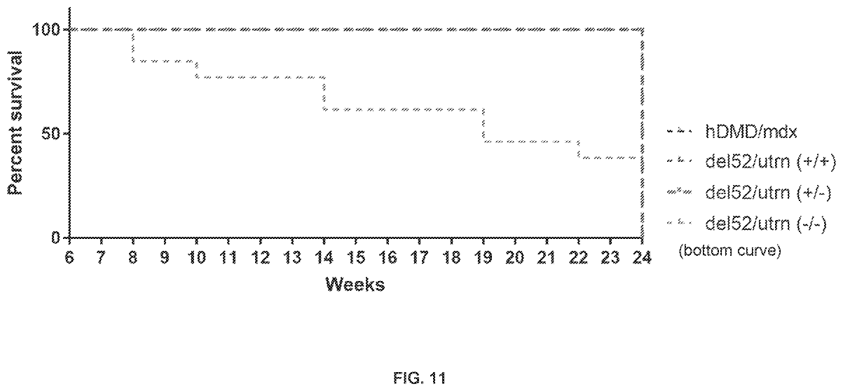

Body mass of hDMDΔ52/mdx/Utrn WT, hDMDΔ52/mdx/Utrn het and hDMDΔ52/mdx/Utrn KO mice was recorded over time (FIG. 10A). Statistical analysis was performed using a two-way ANOVA with Tukey's test (TABLE 4). Muscle mass was recorded at 24 weeks of age (FIG. 10B). These data show that loss of utrophin decreases body and muscle mass of hDMDΔ52/mdx mice.

| Comparison | P value |
|---|---|
| hDMD vs. del52/utrn WT | <0.00001 |
| hDMD vs. del52/utr het | <0.00001 |
| hDMD vs. del52/utr KO | <0.00001 |
| del52/utrn WT vs. del52/utrn het | <0.00001 |
| del52/utrn WT vs. del52/utrn KO | <0.00001 |
| del52/utrn het vs. del52/utrn KO | <0.00001 |

Lifespan was compared amongst the different genotypes. hDMDΔ52/mdx/Utrn KO mice displayed a noticeably shortened lifespan (median survival of ˜19 weeks of age), while other genotypes remained viable over the entire course of the study (FIG. 11).

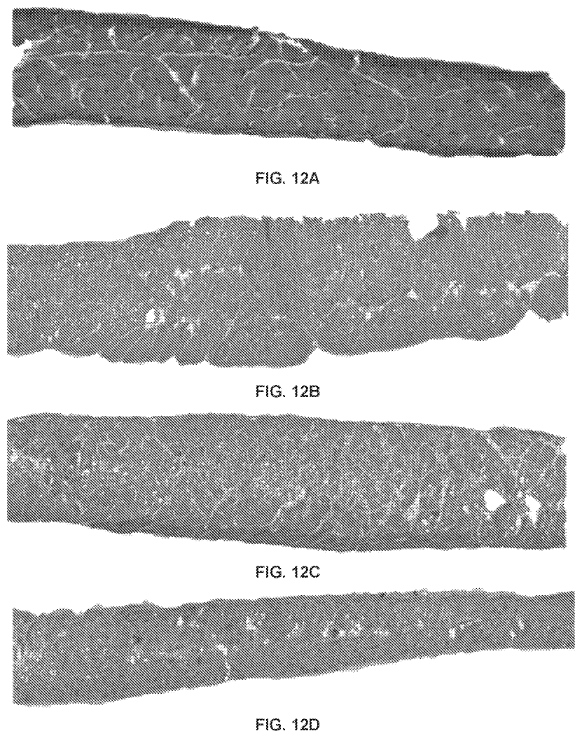

H&E staining was performed to assess dystrophic pathology in diaphragm muscle at 24 weeks of age. Control hDMD/mdx mice displayed normal muscle histology consisting of organized, uniformly sized muscle fibers (pink) with peripheral nuclei (blue) (FIG. 12A). Dystrophic pathology was observed in hDMDΔ52/mdx mice, which is marked by regenerating (smaller) fibers with centralized nuclei, disorganized structure, and immune cell infiltration (punctate, grouped nuclear staining) (FIG. 12B). hDMDΔ52/mdx/Utrn het muscle displayed all of the markers of the dystrophic phenotype—reduction of muscle fibers, increased immune cell infiltration and increased apoptotic fibers (darker, enlarged fibers) (FIG. 12C). hDMDΔ52/mdx/Utrn KO muscle also displayed all of the markers of the dystrophic phenotype, with a noticeable reduction of muscle fibers, increased immune cell infiltration and more apoptotic fibers (darker, enlarged fibers) (FIG. 12D). Thus, demonstrating that loss of utrophin exacerbates muscle degeneration of hDMDΔ52/mdx mice.

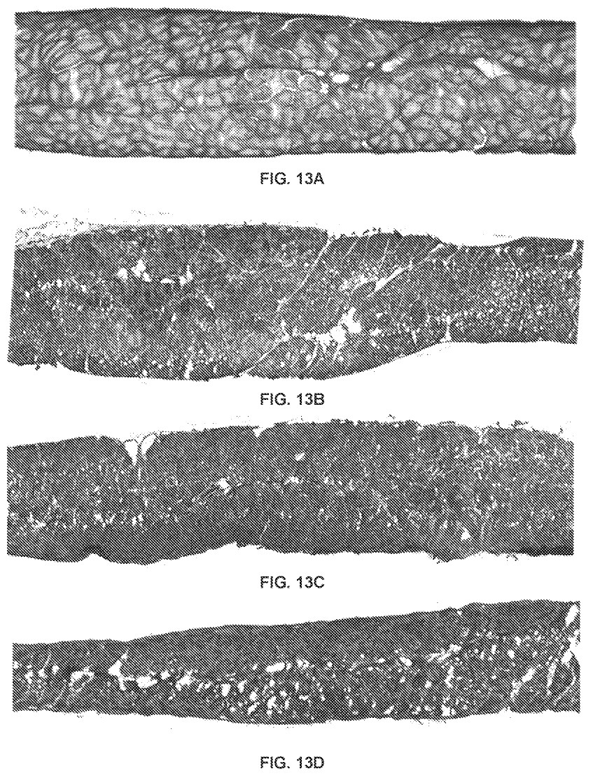

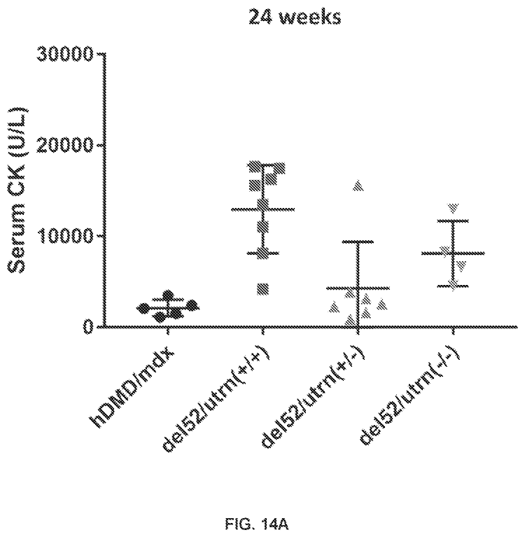

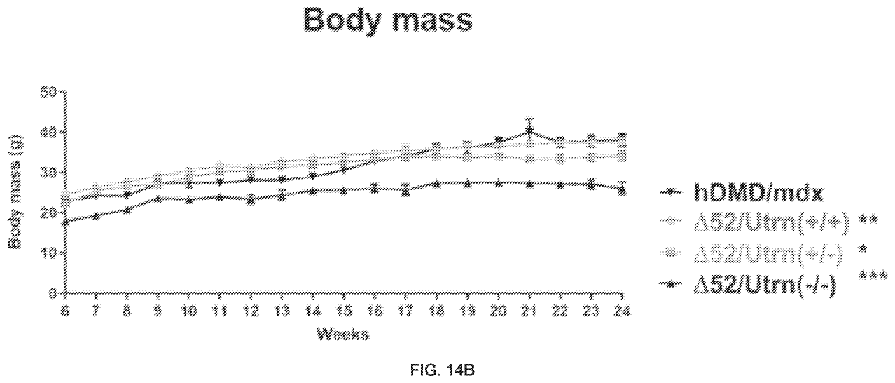

Masson trichrome staining was performed to examine fibrosis in diaphragm muscle at 24 weeks of age. Control hDMD/mdx mice displayed normal muscle histology, consisting of little collagen deposition (blue) surrounding the muscle fibers (red) (FIG. 13A). Increased collagen deposition was observed in hDMDΔ52/mdx mice, replacing a significant portion of muscle mass (FIG. 13B). Fibrotic deposition was highly apparent in hDMDΔ52/mdx/Utrn het (FIG. 13C) and hDMDΔ52/mdx/Utrn KO (FIG. 13D) muscle, appearing to take over more than half of the diaphragm. These data indicate that loss of utrophin increases fibrotic deposition in hDMDΔ52/mdx muscle.

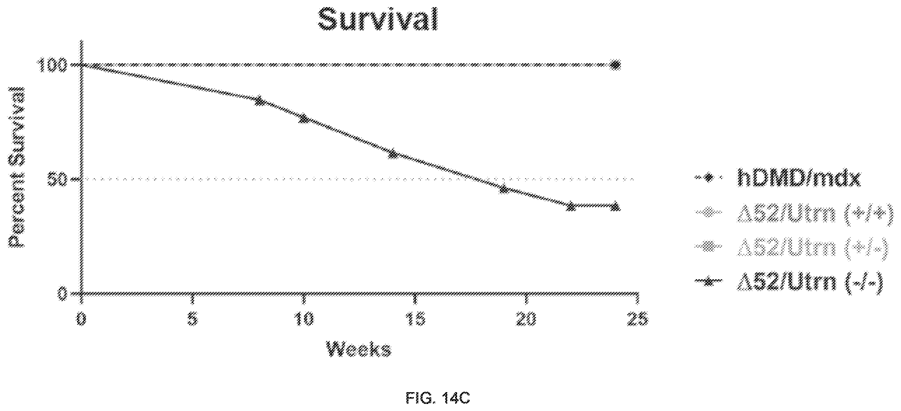

Serum creatine kinase (CK) was measured to assess the level of muscle degeneration in each mouse line at 24 weeks of age. Control hDMD/mdx serum contained low levels of CK, while hDMDΔ52/mdx and hDMDΔ52/mdx/Utrn KO mice contained higher levels of CK, indicative of muscle fiber damage (FIG. 14A). Utrophin-deficient mice exhibited common hallmarks of the dystrophic phenotype for body mass (FIG. 14B) and survival (FIG. 14C). Statistical analysis was performed using a t-test with Welch's correction to compare groups. Therefore, loss of utrophin increases serum biomarker of muscle damage. At 24 weeks, muscle mass was greatly reduced. There was less variability in the hDMDΔ52/mdx/Utrn KO mice overall. Also, many hDMDΔ52/mdx/Utrn KO mice died at this point.

### Example 5

### CRISPR/Cas9 Treatment of hDMDΔ52/mdx/Utrn KO Mice Restores Dystrophin Expression

Skilled artisans will appreciate that changes in genotype and/or phenotype observed in humanized mouse models of DMD can be predictive of changes in genotype and/or phenotype in human patients treated with the compositions and methods of the present disclosure. In particular, a method or composition that is efficacious in rescuing a disease (or disease-like) genotype or phenotype in a humanized mouse model can be readily adapted by those of skill in the art to therapeutic use in human subjects, and such adaptations are within the scope of the present disclosure.

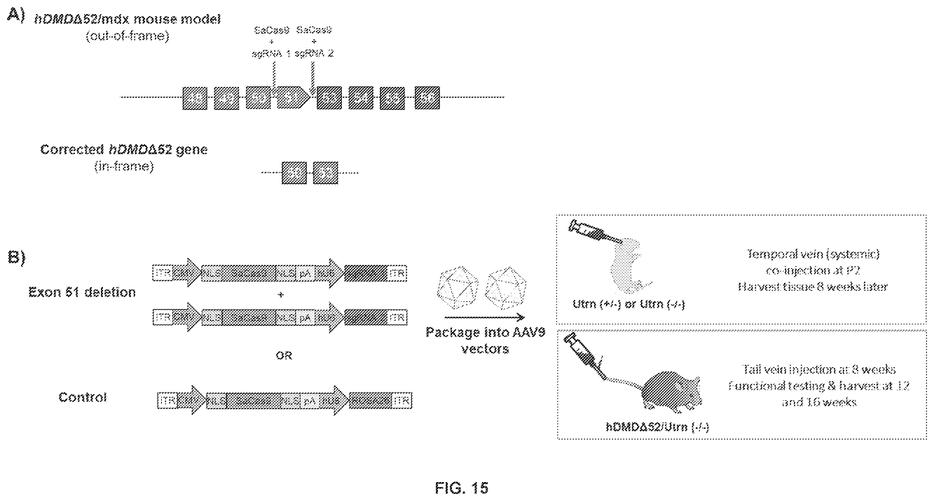

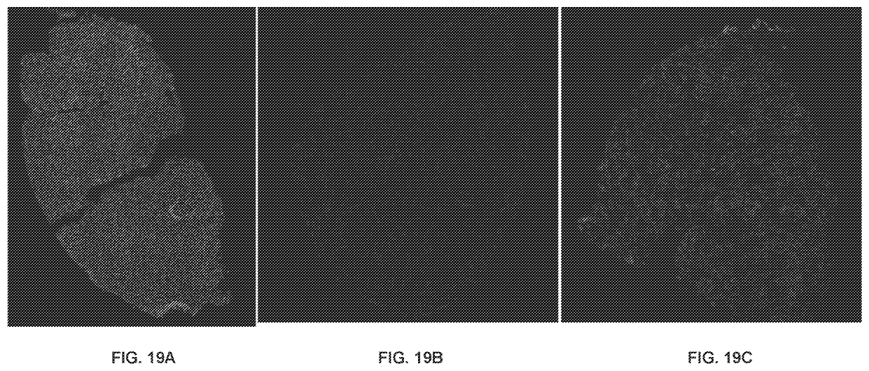

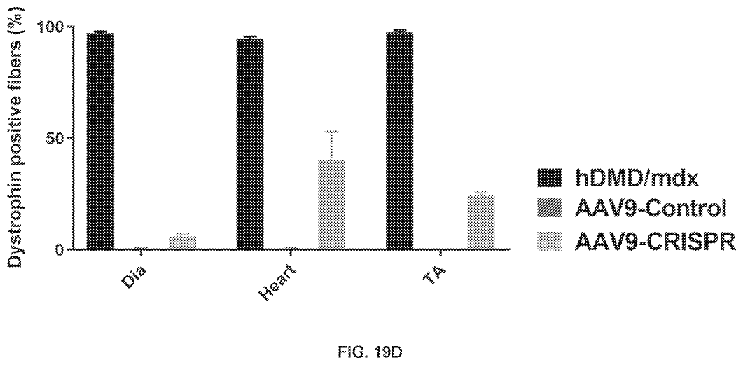

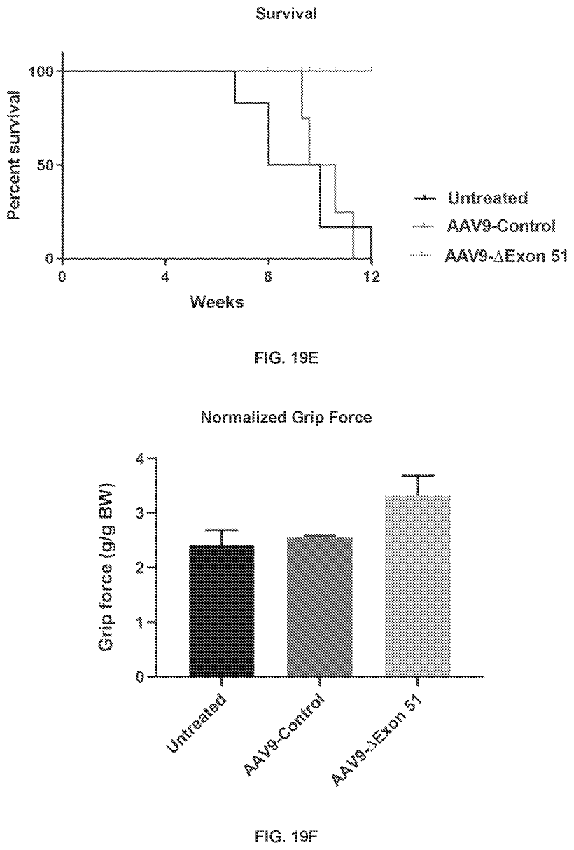

Neonates and adults were injected to test effect of CRISPR/Cas9 treatment on pathology and phenotypic rescue in Utrn deficient lines and compare the phenotypic improvements in treated Utrn deficient mice (FIG. 15). Neonatal Utrn hets and Utrn KOs were treated with 7.5×10¹¹ total vector genomes of either the AAV9-ROSA26 control or AAV9-ΔExon 51 via temporal vein injection. Adult Utrn KOs were treated with 4×10¹² total vector genomes of either the AAV9-ROSA26 control or AAV9-ΔExon 51 via tail vein injection. FIG. 19A shows muscle from the age-matched hDMD/mdx wild-type control for comparison to Utrn KO mice treated as adults with AAV9-ROSA26 control (FIG. 19B) or AAV9-ΔExon 51 (FIG. 19C). The gRNAs are shown in TABLE 5, wherein the sequences shown are those of the DNA target the gRNA binds and targets.

TABLE 5

| Name | Description | Sequence |
|---|---|---|
| JCR179 | Intron 50 guide | AACACACAGCTGGGTTATCAGAG |
| | | (SEQ ID NO: 53) |
| JCR183 | Intron 51 guide | GAACTGGTGGGAAATGGTCTAG |
| | | (SEQ ID NO: 54) |

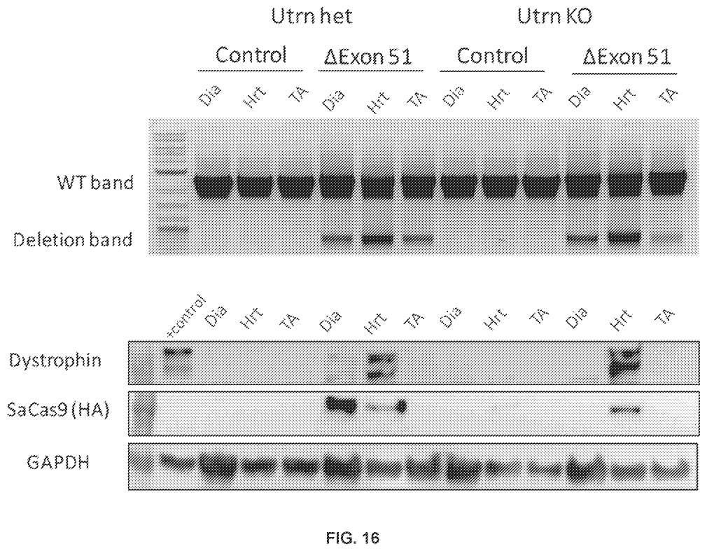

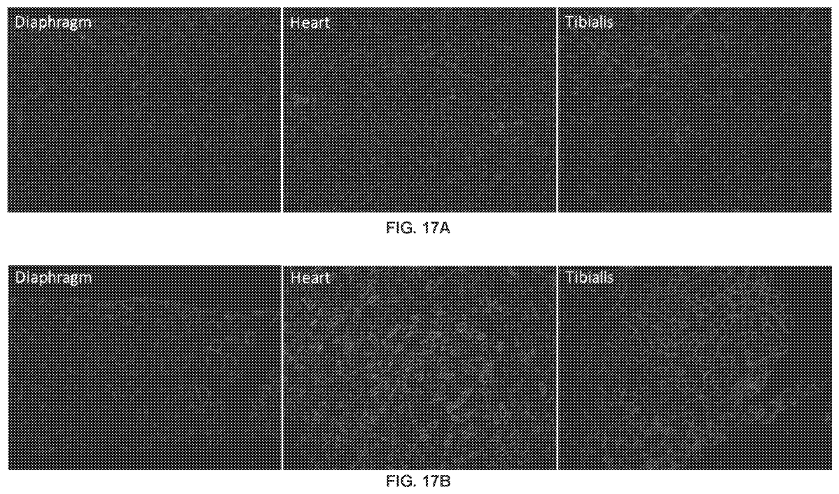

After CRISPR treatment, deletion PCR of genomic DNA was performed to determine if Exon 51 deletion was achieved (FIG. 16, top). Compared to the controls, CRISPR-ΔExon 51 treatment resulted in excision of exon 51 in both hDMDΔ52/mdx/Utrn het and hDMDΔ52/mdx/Utrn KO mice. Western blotting was performed to measure dystrophin expression in treated mice (FIG. 16, bottom). Dystrophin expression was apparent in both mouse lines after treatment with CRISPR-ΔExon 51.

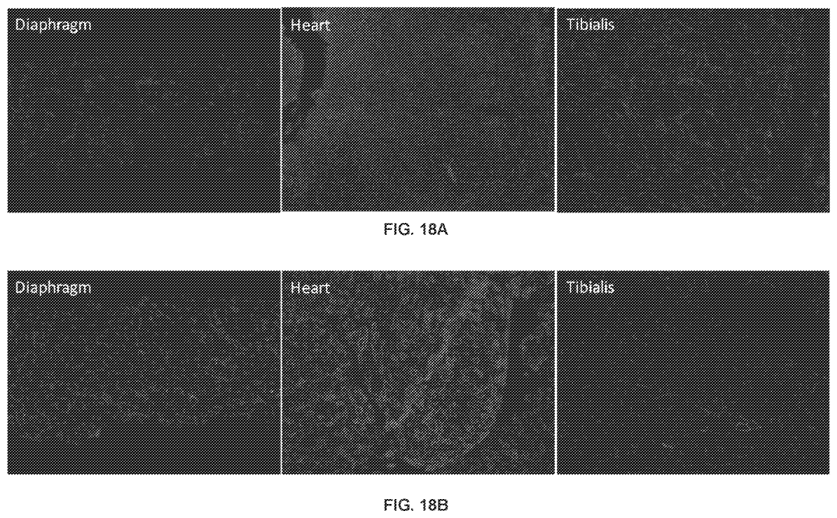

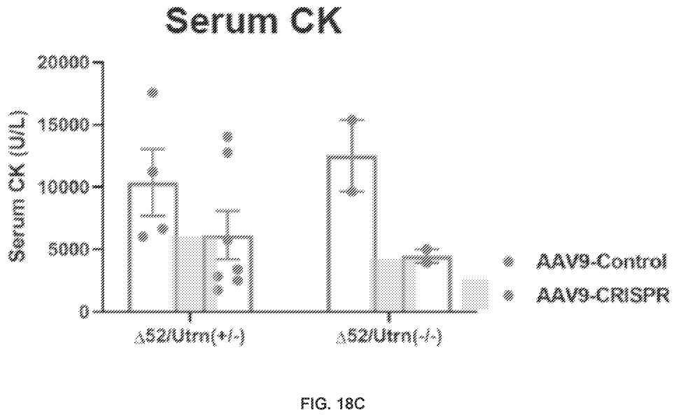

After CRISPR treatment, immunofluorescent staining was performed. Compared to the controls, CRISPR-ΔExon 51 treatment resulted in restoration of dystrophin (red) in the muscles of both hDMDΔ52/mdx/Utrn het and hDMDΔ52/mdx/Utrn KO mice (FIG. 17A, FIG. 17B, FIG. 18A, and FIG. 18B). CRISPR-ΔExon 51 treatment also resulted in reduced serum creatine kinasae (CK) in both hDMDΔ52/mdx/Utrn het and hDMDΔ52/mdx/Utrn KO mice (FIG. 18C). Confirmation of dystrophin restoration coupled with the functional aspects of hDMDΔ52/mdx/Utrn KO mice led us to continue our studies in these mice. Adult mice hDMDΔ52/mdx/Utrn KO mice were treated at 8 weeks of age with a control vector or CRISPR-ΔExon 51 (FIG. 19A, FIG. 19B, FIG. 19C, and FIG. 19D). Immunofluorescent staining of the tibialis anterior muscle at 16 weeks post-treatment revealed widespread dystrophin staining in CRISPR-ΔExon 51-treated mice (FIG. 19C). Dystrophin positive fibers were also quantified (FIG. 19D). Exon 51 deletion improved survival (FIG. 19E) and motor function (FIG. 19F) in Utrn KO mice.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of screening for a pair of gRNA molecules for editing a genomic nucleic acid in a subject, the method comprising: (a) generating a plurality of pairs of gRNA molecules, each pair comprising a first gRNA and a second gRNA, wherein the first gRNA targets a first nucleic acid sequence and the second gRNA targets a second nucleic acid sequence; (b) expressing a Cas9 protein or a fusion protein comprising the Cas9 protein, and the plurality of pairs of gRNA molecules in a plurality of cells, wherein one pair of gRNA molecules is expressed in a cell, and wherein the first gRNA directs the Cas9 protein or fusion protein to cut the first nucleic acid sequence and the second gRNA directs the Cas9 protein or fusion protein to cut the second nucleic acid sequence.

Clause 2. The method of clause 1, wherein expressing the Cas9 protein or the fusion protein comprising the Cas9 protein, and the plurality of pairs of gRNA molecules in the plurality of cells, wherein one pair of gRNA molecules is expressed in a cell, and wherein the first gRNA directs the Cas9 protein or fusion protein to cut the first nucleic acid sequence and the second gRNA directs the Cas9 protein or fusion protein to cut the second nucleic acid sequence in step (b), thereby forms an excised nucleic acid and a new junction in the genomic nucleic acid.

Clause 3. The method of clause 2, wherein the excised nucleic acid is in-frame.

Clause 4. The method of any one of clauses 1-3, wherein the genomic nucleic acid comprises at least one exon of a dystrophin gene, wherein the first nucleic acid sequence comprises a first intron of the dystrophin gene and the second nucleic acid sequence comprises a second intron of the dystrophin gene, and wherein the first intron is adjacent to one side of the at least one exon and the second intron is adjacent to the other side of the at least one exon.

Clause 5. The method of clause 4, wherein the at least one exon is in between the first and second introns in the genomic nucleic acid.

Clause 6. The method of any one of clauses 1-5, wherein the genomic nucleic acid comprises two or more exons of a dystrophin gene, wherein the first nucleic acid sequence comprises a first intron of the dystrophin gene and the second nucleic acid sequence comprises a second intron of the dystrophin gene, and wherein the first intron is adjacent to one side of the two or more exons and the second intron is adjacent to the other side of the two or more exons.

Clause 7. The method of clause 6, wherein the two or more exons are in between the first and second introns in the genomic nucleic acid.

Clause 8. The method of any one of clauses 1-7, wherein the expression is effected by transfecting the plurality of cells with a plurality of vectors, wherein each cell is transfected with a first vector encoding one pair of gRNA molecules and a second vector encoding the Cas9 protein or fusion protein, wherein each cell is transfected with a different first vector encoding a different pair of gRNA molecules.

Clause 9. The method of clause 8, wherein the first vector and second vector are each a viral vector.

Clause 10. The method of clause 9, wherein the viral vector is a lentiviral vector, a AAV vector, or an adenoviral vector.

Clause 11. The method of any one of clauses 1-10, the method further comprising: (c) isolating the genomic nucleic acid from the plurality of cells; and/or (d) contacting the genomic nucleic acid with a first pool of probes, wherein one or more different probes specifically bind to each new junction and a portion of the first nucleic acid sequence; and/or (e) isolating the genomic nucleic acid bound to the first pool of probes; and/or (f) contacting the genomic nucleic acid bound to the first pool of probes with a second pool of probes, wherein one or more different probes specifically bind to each new junction and a portion of the second nucleic acid sequence; and/or (g) isolating the genomic nucleic acid bound to the first and second pools of probes; and/or (h) sequencing the isolated genomic nucleic acid bound to the first and second pools of probes; and/or (i) aligning the sequenced isolated genomic nucleic acid to identify the sequenced new junctions; and/or (j) assigning each sequenced new junction to the corresponding pair of gRNA molecules.

Clause 12. The method of clause 11, wherein step (i) comprises computationally aligning the sequences of the isolated genomic nucleic acid to identify the sequenced new junctions.

Clause 13. The method of clause 12 or 13, further comprising identifying the pair of gRNA molecules having a greater number of sequenced new junctions as the pair of gRNA molecules having greater efficiency.

Clause 14. The method of any one of clauses 11-13, wherein the probes each have a length of about 100 bp to about 140 bp.

Clause 15. The method of any one of clauses 1-14, wherein the excised nucleic acid comprises exon 51 of the dystrophin gene.

Clause 16. The method of any one of clauses 1-15, wherein the excised nucleic acid comprises exons 45-55 of the dystrophin gene.

Clause 17. The method of any one of clauses 1-15, wherein the first nucleic acid sequence is within intron 50 of the dystrophin gene.

Clause 18. The method of any one of clauses 1-15, wherein the second nucleic acid sequence is within intron 51 of the dystrophin gene.

Clause 19. The method of any one of clauses 1-16, wherein the first nucleic acid sequence is within intron 44 of the dystrophin gene.

Clause 20. The method of any one of clauses 1-16, wherein the second nucleic acid sequence is within intron 55 of the dystrophin gene.

Clause 21. The method of any one of clauses 1-20, wherein the probes are biotinylated probes.

Clause 22. A pair of gRNA molecules identified by the method of any one of the preceding clauses.

Clause 23. A CRISPR/Cas9 system comprising the pair of gRNA molecules of clause 22.

Clause 24. A gRNA molecule that binds and targets a polynucleotide sequence, and wherein the gRNA molecule binds or is encoded by a polynucleotide comprising a sequence selected from SEQ ID NOs: 55-78, or wherein the gRNA molecule comprises a polynucleotide sequence selected from SEQ ID NOs: 79-102.

Clause 25. A transgenic mouse whose genome comprises: a mutation in the mouse dystrophin gene: a mutant human dystrophin gene on chromosome 5; and a mutation in the mouse utrophin gene.

Clause 26. The mouse of clause 25, wherein the mutation in the mouse dystrophin gene comprises an insertion or deletion in the mouse dystrophin gene that prevents protein expression from the mouse dystrophin gene.

Clause 27. The mouse of clause 26, wherein the mutation in the mouse dystrophin gene comprises a premature stop codon in exon 23 of the mouse dystrophin gene.

Clause 28. The mouse of any one of clauses 25-27, wherein the mutant human dystrophin gene has at least one exon deleted.

Clause 29. The mouse of any one of clauses 25-28, wherein the mutant human dystrophin gene has exon 52 deleted.

Clause 30. The mouse of any one of clauses 25-29, wherein the mutation in the mouse utrophin gene is a functional deletion of the mouse utrophin gene.

Clause 31. The mouse of any one of clauses 25-29, wherein the mutation in the mouse utrophin gene comprises an insertion or deletion in the mouse utrophin gene that prevents protein expression from the mouse utrophin gene.

Clause 32. The mouse of clause 31, wherein the mutation in the mouse utrophin gene comprises an insertion in exon 7 of the mouse utrophin gene.

Clause 33. The mouse of any one of clauses 25-29, wherein the mutation in the mouse utrophin gene comprises a deletion of the entire mouse utrophin gene.

Clause 34. The mouse of any one of clauses 25-33, wherein the mouse is heterozygous for the mutation in the mouse utrophin gene.

Clause 35. The mouse of any one of clauses 25-33, wherein the mouse is homozygous for the mutation in the mouse utrophin gene.

Clause 36. The mouse of any one of clauses 25-35, wherein the mouse has reduced life span, reduced body mass, reduced body strength, reduced motor coordination, reduced balance, and/or reduced forelimb strength as compared to a wild-type mouse.

Clause 37. The mouse of any one of clauses 25-35, wherein the mouse has reduced life span, reduced body mass, reduced body strength, reduced motor coordination, reduced balance, and/or reduced forelimb strength as compared to a control mouse whose genome comprises a wild-type utrophin gene and a mutation in the mouse dystrophin gene.

Clause 38. The mouse of any one of clauses 25-35, wherein the mouse has reduced lifespan, reduced body mass, reduced body strength, reduced motor coordination, reduced balance, and/or reduced forelimb strength as compared to a control mouse whose genome comprises a wild-type utrophin gene, a mutation in the mouse dystrophin gene, and a mutant human dystrophin gene.

Clause 39. The mouse of any of clauses 25-38, wherein the mouse has increased muscle damage as compared to (i) a wild-type mouse, (ii) a control mouse whose genome comprises a wild-type utrophin gene and a mutation in the mouse dystrophin gene, and/or (iii) a control mouse whose genome comprises a wild-type utrophin gene, a mutation in the mouse dystrophin gene, and a mutant human dystrophin gene.

Clause 40. The mouse of clause 39, wherein the muscle damage comprises one or more of degeneration of the muscle, fibrosis of the muscle, and elevated serum creatine kinase.

Clause 41. The mouse of any one of clauses 25-40, wherein the mouse does not exhibit detectable dystrophin protein in heart or skeletal muscle.

Clause 42. The mouse of any one of clauses 25-41, wherein the mouse is a hDMDΔ52/mdx/Utrn KO mouse.

Clause 43. An isolated cell or biological material obtained from the mouse of any one of clauses 25-42.

Clause 44. The biological material of clause 43, comprising a protein, a lipid, a nucleotide, fat, muscle, or a tissue.

Clause 45. A method of correcting a dystrophin gene mutation, the method comprising administering to the mouse of any one of clauses 25-42 a CRISPR/Cas9 gene editing composition.

Clause 46. The method of clause 45, wherein the CRISPR/Cas9 gene editing composition comprises: (a) at least one guide RNA (gRNA) targeting the mutant human dystrophin gene; and (b) a Cas9 protein or a fusion protein comprising the Cas9 protein.

Clause 47. The method of clause 46, wherein the CRISPR/Cas9 gene editing composition comprises a first gRNA and a second gRNA, and wherein the first gRNA and the second gRNA are configured to form a first and a second double strand break in a first and a second intron flanking exon 51 of the mutant human dystrophin gene, respectively, thereby deleting exon 51.

Clause 48. The method of clause 47, wherein the CRISPR/Cas9 gene editing composition comprises a first gRNA and a second gRNA, and wherein the first gRNA and the second gRNA are configured to form a first and a second double strand break in a first and a second intron flanking exons 45-55 of the mutant human dystrophin gene, respectively, thereby deleting exons 45-55.

Clause 49. The method of any one of clauses 45-48, wherein the dystrophin gene mutation is corrected in a cell of the mouse, and wherein the cell is a muscle cell, a satellite cell, or an iPSC/iCM.

Clause 50. The method of any one of clauses 45-49, wherein the correction restores the reading frame of the human dystrophin gene.

Clause 51. The method of any one of clauses 45-50, wherein the correction results in expression of an at least partially functional human dystrophin protein.

Clause 52. A gamete produced by the mouse of any one of clauses 25-42.

Clause 53. The gamete of clause 52, wherein the gamete does not encode a functional mouse dystrophin protein or a functional mouse utrophin protein.

Clause 54. An isolated mouse cell, or a progeny cell thereof, isolated from the mouse of any one of clauses 25-42.

Clause 55. A primary cell culture or a secondary cell line derived from the mouse of any one of clauses 25-42.

Clause 56. A tissue or organ explant or culture thereof, derived from the mouse of any one of clauses 25-42

Clause 57. A method of screening therapeutic agents for treating Duchenne muscular dystrophy (DMD), the method comprising administering to the mouse of any one of clauses 25-42 one or more therapeutic agents.

Clause 58. The method of clause 57, wherein the one or more therapeutic agents comprises a small molecule, anti-sense RNA, vector, CRISPR/Cas gene editing system, or biological agent, or a combination thereof.

Clause 59. The method of clause 58, wherein the vector is a viral vector encoding a gene of interest.

Clause 60. The method of clause 59, wherein the viral vector is an AAV vector.

Clause 61. The method of any one of clauses 57-60, wherein the mouse after administration of the one or more therapeutic agents exhibits increased lifespan, reduced body mass, increased body strength, increased motor coordination, increased balance, increased forelimb strength, reduced muscle injury, and/or reduced CK level compared to before administration of the one or more therapeutic agents.

Clause 62. The method of any one of clauses 57-61, wherein the mouse after administration of the one or more therapeutic agents exhibits increased expression of a dystrophin gene as compared to before administration of the one or more therapeutic agents.

Clause 63. The method of clause 62, wherein the dystrophin gene is a truncated human dystrophin gene.

Clause 64. The method of clause 63, wherein the truncated human dystrophin gene comprises a plurality of deletions relative to a wild-type human dystrophin gene, and wherein at least one of the deletions is in exon 52.

SEQUENCES

SEQ ID NO: 1
NRG (N can be any nucleotide residue, e.g., any of A, G, C, or T)
SEQ ID NO: 2
NGG (N can be any nucleotide residue, e.g., any of A, G, C, or T)
SEQ ID NO: 3
NAG (N can be any nucleotide residue, e.g., any of A, G, C, or T)
SEQ ID NO: 4
NGGNG (N can be any nucleotide residue, e.g., any of A, G, C, or T)
SEQ ID NO: 5
NNAGAAW (W = A or T; N can be any nucleotide residue, e.g., any of A, G, C, or T)
SEQ ID NO: 6
NAAR (R = A or G; N can be any nucleotide residue, e.g., any of A, G, C, or T)
SEQ ID NO: 7
NNGRR (R = A or G; N can be any nucleotide residue, e.g., any of A, G, C, or T)
SEQ ID NO: 8
NNGRRN (R = A or G; N can be any nucleotide residue, e.g., any of A, G, C, or T)
SEQ ID NO: 9
NNGRRT (R = A or G; N can be any nucleotide residue, e.g., any of A, G, C, or T)
SEQ ID NO: 10
NNGRRV (R = A or G; N can be any nucleotide residue, e.g., any of A, G, C, or T)
SEQ ID NO: 11
NNNNGATT (N can be any nucleotide residue, e.g., any of A, G, C, or T)
SEQ ID NO: 12
NNNNGNNN (N can be any nucleotide residue, e.g., any of A, G, C, or T)
SEQ ID NO: 13
NGA (N can be any nucleotide residue, e.g., any of A, G, C, or T)
SEQ ID NO: 14
NNNRRT (R = A or G; N can be any nucleotide residue, e.g., any of A, G, C, or T)
SEQ ID NO: 15
ATTCCT
SEQ ID NO: 16
NGAN (N can be any nucleotide residue, e.g., any of A, G, C, or T)
SEQ ID NO: 17
NGNG (N can be any nucleotide residue, e.g., any of A, G, C, or T)
SEQ ID NO: 18
Streptococcus pyogenes Cas9 protein
MDKKYSIGLDIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKERGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLEKTNRKVTVKQLKEDYFKKIECFDSVEISGVEDRENASLGTYHDLLKIIKDKDELDNEEN
EDILEDIVLTLTLFEDREMIEERLKTYAHLEDDKVMKQLKERRYTGWGRLSRKLINGIRDKQSGKTIL
DELKSDGFANRNEMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLINAKLITQRKEDNLTKAERGGLSELDKAGFIKRQLVETRQITKHVAQILDSRMNTKYDENDKLIRE
VKVITLKSKLVSDERKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 19
Staphylococcus aureus Cas9 protein
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
TIENAELLDQIAKILTIYQSSEDIQEELININSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSEDNSENNKVLVKQEENSKKGNRTPFQYLSSSDSKISYETFKKHILNLA
KGKGRISKTKKEYLLEERDINRESVQKDEINENLVDTRYATRGLMNLLRSYFRVNNLDVKVKSINGGF
ISFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKEVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
SEQ ID NO: 20
Streptococcus pyogenes Cas9 amino acid sequence (with D10A)
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLEKTNRKVTVKQLKEDYFKKIECFDSVEISGVEDRENASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKEDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLEITNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 21
Streptococcus pyogenes Cas9 amino acid sequence (with D10A, H849A)
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLEKTNRKVTVKQLKEDYFKKIECFDSVEISGVEDRENASLGTYHDLLKIIKDKDFLDNEEN
EDILEDIVITLTLFEDREMIEERLKTYAHLEDDKVMKQLKRRRYTGWGRLSRKLINGIRDKQSGKTIL
DELKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLEVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 22
Polynucleotide sequence of D10A mutant of S. aureus Cas9
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 23
Polynucleotide sequence of N580A mutant of S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 24
codon optimized polynucleotide encoding S. pyogenes Cas9
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg agcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tctggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tccaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacc acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tcggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga aacacggatc
gacctctctc aactgggcgg cgactag
SEQ ID NO: 25
codon optimized nucleic acid sequences encoding S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa agccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 26
codon optimized nucleic acid sequences encoding S. aureus Cas9
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgtac
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
SEQ ID NO: 27
codon optimized nucleic acid sequence encoding S. aureus Cas9
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
SEQ ID NO: 28
codon optimized nucleic acid sequence encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 29
codon optimized nucleic acid sequence encoding S. aureus Cas9
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
tcc
SEQ ID NO: 30
codon optimized nucleic acid sequence encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 31
codon optimized nucleic acid sequences encoding S. aureus Cas9
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
SEQ ID NO: 32
Vector (pDO242) encoding codon optimized nucleic acid sequence encoding
S. aureus Cas9
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGOTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
SEQ ID NO: 33
Human p300 (with L553M mutation) protein
MAENVVEPGPPSAKRPKLSSPALSASASDGTDFGSLEDLEHDLPDELINSTELGLINGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSQAQMSSSSCPVNSPIMPPGSQGSH
IHCPQLPQPALHQNSPSPVPSRTPTPHHTPPSIGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMFNNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKNDTLDPELFVECTECGRKMHQICVLHHEIIWPAGEVCDGCLKKSARTRKENKESAKR
LPSTRLGTFLENRVNDFLRRQNHPESGEVTVRVVHASDKTVEVKPGMKAREVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDREVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
RIGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
SEQ ID NO: 34
Human p300 Core Effector protein (aa 1048-1664 of SEQ ID NO: 33)
IFKPEELRQALMPTLEALYRQDPESLPFRQPVDPQLLGIPDYFDIVKSPMDLSTIKRKLDTGQYQEPW
QYVDDIWLMENNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELEVECTECG
RKMHQICVLHHEIIWPAGEVCDGCLKKSARTRKENKFSAKRLPSTRLGTFLENRVNDFLRRQNHPESG
EVTVRVVHASDKTVEVKPGMKAREVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDEWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
SEQ ID NO: 35
VP64-dCas9-VP64 protein
RADALDDFDLDMLGSDALDDFDLDMLGSDALDDFDLDMLGSDALDDFDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESELVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLEKTNRKVTVKQLKEDYFKKIECFDSVEISGVEDRENASLGTYHDLLKIIKDKDFLDNEENEDILE
DIVLTLTLFEDREMIEERLKTYAHLEDDKVMKQLKRRRYTGWGRLSRKLINGIRDKQSGKTILDELKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLEVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLSQL
GGDSRADPKKKRKVASRADALDDFDLDMLGSDALDDFDLDMLGSDALDDFDLDMLGSDALDDFDLDML
I
SEQ ID NO: 36
VP64-dCas9-VP64 DNA
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
SEQ ID NO: 37
mouse utrophin DNA sequence
SEQ ID NO: 38
mouse utrophin amino acid sequence
MAKYGDLEARPDDGQNEFSDIIKSRSDEHNDVQKKTFTKWINARFSKSGKPPISDMFSDL
KDGRKLLDLLEGLTGTSLPKERGSTRVHALNNVNRVLQVLHQNNVDLVNIGGTDIVDGNP
KLTLGLLWSIILHWQVKDVMKDIMSDLQQTNSEKILLSWVRQTTRPYSQVNVLNETTSWT
DGLAFNAVLHRHKPDLESWDRVVKMSPIERLEHAFSKAHTYLGIEKLLDPEDVAVHLPDK
KSIIMYLTSLFEVLPQQVTIDAIREVETLPRKYKKECEEEEIHIQSAVLAEEGQSPRAET
PSTVTEVDMDLDSYQIALEEVLTWLLSAEDTFQEQDDISDDVEEVKEQFATHETFMMELT
AHQSSVGSVLQAGNQLMTQGTLSEEEEFEIQEQMTLLNARWEALRVESMERQSRLHDALM
ELQKKQLQQLSSWLALTEERIQKMESLPLGDDLPSLQKLLQEHKSLQNDLEAEQVKVNSL
THMVVIVDENSGESATALLEDQLQKLGERWTAVCRWTEERWNRLQEISILWQELLEEQCL
LEAWLTEKEEALNKVQTSNFKDQKELSVSVRRLAILKEDMEMKRQTLDQLSEIGQDVGQL
LSNPKASKKMNSDSEELTQRWDSLVQRLEDSSNQVTQAVAKLGMSQIPQKDLLETVHVRE
QGMVKKPKQELPPPPPPKKRQIHVDVEAKKKFDAISTELLNWILKSKTAIQNTEMKEYKK
SQETSGMKKKLKGLEKEQKENLPRLDELNQTGQTLREQMGKEGLSTEEVNDVLERVSLEW
KMISQQLEDLGRKIQLQEDINAYFKQLDAIEETIKEKEEWLRGTPISESPRQPLPGLKDS
CQRELTDLLGLHPRIETLCASCSALKSQPCVPGFVQQGFDDLRHHYQAVRKALEEYQQQL
ENELKSQPGPAYLDTLNTLKKMLSESEKAAQASLNALNDPIAVEQALQEKKALDETLENQ
KHTLHKLSEETKTLEKNMLPDVGKMYKQEFDDVQGRWNKVKTKVSRDLHLLEEITPRLRD
FEADSEVIEKWVSGIKDELMKEQAAQGDAAALQSQLDQCATFANEIETIESSLKNMREVE
TSLQRCPVTGVKTWVQARLVDYQSQLEKFSKEIAIQKSRLSDSQEKALNLKKDLAEMQEW
MAQAEEDYLERDFEYKSPEELESAVEEMKRAKEEVLQKEVRVKILKDSIKLVAAKVPSGG
QELTSEFNEVLESYQLLCNRIRGKCHTLEEVWSCWVELLHYLDLETTWLNTLEERVRSTE
ALPERAEAVHEALESLESVLRHPADNRTQIRELGQTLIDGGILDDIISEKLEAFNSRYEE
LSHLAESKQISLEKQLQVLRETDHMLQVLKESLGELDKQLTTYLTDRIDAFQLPQEAQKI
QAEISAHELTLEELRKNVRSQPPTSPEGRATRGGSQMDMLQRKLREVSTKFQLFQKPANE
EQRMLDCKRVLEGVKAELHVLDVRDVDPDVIQAHLDKCMKLYKTLSEVKLEVETVIKTGR
HIVQKQQTDNPKSMDEQLTSLKVLYNDLGAQVTEGKQDLERASQLSRKMKKEAAVLSEWL
SATEAELVQKSTSEGVIGDLDTEISWAKSILKDLEKRKVDLNGITESSAALQHLVLGSES
VLEENLCVLNAGWSRVRTWTEDWQNTLLNHQNQLELFDGHVAHISTWLYQAEALLDEIEK
KPASKQEEIVKRLLSELDDASLQVENVREQAIILVNARGSASRELVEPKLAELSRNFEKV
SQHIKSARMLIGQDPSSYQGLDPAGTVQAAESFSDLENLEQDIENMLKVVEKHLDPNNDE
KMDEEQAQIEEVLQRGEHLLHEPMEDSKKEKIRLQLLLLHTRYNKIKTIPIQQRKTIPVS
SGITSSALPADYLVEINKILLTLDDIELSLNMPELNTTVYKDFSFQEDSLKSIKGQLDRL
GEQIAVVHEKQPDVIVEASGPEAIQIRDMLAQLNAKWDRVNRVYSDRRGSFARAVEEWRQ
FHHDLDDLTQWLSEAEDLLVDTCAPDGSLDLEKARAQQLELEEGLSSHQPSLIKVNRKGE
DLVQRLRPSEASFLKEKLAGENQRWSTLVAEVEALQPRLKGESQQVLGYKRRLDEVTCWL
TKVESAVQKRSTPDPEESPQELTDLAQETEVQAENIKWINRAELEMLSDKNLSLREREKL
SESLRNVNTTWTKVCREVPSLLKTRTQDPCSAPQMRMAAHPNVQKVVLVSSASDAPLRGG
LEISVPADLDKTITELADWLVLIDQMLKSNIVTVGDVKEINKTVSRMKITKADLEQRHPQ
LDCVFTLAQNLKNKASSSDVRTAITEKLEKLKTQWESTQHGVELRRQQLEDMVVDSLQWD
DHREETEELMRKYEARFYMLQQARRDPLSKQVSDNQLLLQELGSGDGVIMAFDNVLQKLL
EEYSGDDTRNVEETTEYLKTSWVNLKQSIADRQSALEAELQTVQTSRRDLENFVKWLQEA
ETTANVLADASQRENALQDSVLARQLRQQMLDIQAEIDAHNDIFKSIDGNRQKMVKALGN
SEEATMLQHRLDDMNQRWNDLKAKSASIRAHLEASAEKWNRLLASLEELIKWINMKDEEL
KKQMPIGGDVPALQLQYDHCKVLRRELKEKEYSVLNAVDQARVFLADQPIEAPEEPRRNP
QSKTELTPEERAQKIAKAMRKQSSEVREKWENLNAVTSNWQKQVGKALEKLRDLQGAMDD
LDADMKEVEAVRNGWKPVGDLLIDSLQDHIEKTLAFREEIAPINLKVKTMNDLSSQLSPL
DLHPSLKMSRQLDDLNMRWKLLQVSVDDRLKQLWEAHRDFGPSSQHFLSTSVQLPWQRSI
SHNKVPYYINHQTQTTCWDHPKMTELFQSLADLNNVRFSAYRTAIKIRRLQKALCLDLLE
LNTTNEVFKQHKLNQNDQLLSVPDVINCLTTTYDGLEQLHKDLVNVPLCVDMCLNWLLVN
YDTGRTGKIRVQSLKIGLMSLSKGLLEEKYRCLEKEVAGPTEMCDQRQLGLLLHDAIQIP
RQLGEVAAFGGSNIEPSVRSCFQQNNNKPEISVKEFIDWMHLEPQSMVWLPVLHRVAAAE
TAKHQAKCNICKECPIVGFRYRSLKHENYDVCQSCFFSGRTAKGHKLHYPMVEYCIPTTS
GEDVRDETKVLKNKFRSKKYFAKHPRLGYLPVQTVLEGDNLETPITLISMWPEHYDPSQS
PQLFHDDTHSRIEQYATRLAQMERTNGSFLTDSSSTIGSVEDEHALIQQYCQTLGGESPV
SQPQSPAQILKSVEREERGELERIIADLEEEQRNLQVEYEQLKEQHLRRGLPVGSPPDSI
VSPHHTSEDSELIAEAKLLRQHKGRLEARMQILEDHNKQLESQLHRLRQLLEQPDSDSRI
NGVSPWASPQHSALSYSLDTDPGPQFHQAASEDLLAPPHDTSTDLTDVMEQINSTEPSCS
SNVPSRPQAM
SEQ ID NO: 39
SV40 NLS
Pro-Lys-Lys-Lys-Arg-Lys-Val
SEQ ID NO: 40
GS linker (Gly-Gly-Gly-Gly-Ser)ₙ, wherein n is an integer between 0 and 10
SEQ ID NO: 41
Gly-Gly-Gly-Gly-Gly
SEQ ID NO: 42
Gly-Gly-Ala-Gly-Gly
SEQ ID NO: 43
Gly-Gly-Gly-Gly-Ser-Ser-Ser
SEQ ID NO: 44
Gly-Gly-Gly-Gly-Ala-Ala-Ala
SEQ ID NO: 45
Streptococcus pyogenes dCas9-KRAB polynucleotide sequence
atggactacaaagaccatgacggtgattataaagatcatgacatcgattacaaggatgacgatgacaa
gatggcccccaagaagaagaggaaggtgggccgcggaatggacaagaagtactccattgggctcgcca
tcggcacaaacagcgtcggctgggccgtcattacggacgagtacaaggtgccgagcaaaaaattcaaa
gttctgggcaataccgatcgccacagcataaagaagaacctcattggcgccctcctgttcgactccgg
ggaaaccgccgaagccacgcggctcaaaagaacagcacggcgcagatatacccgcagaaagaatcgga
tctgctacctgcaggagatctttagtaatgagatggctaaggtggatgactctttcttccataggctg
gaggagtcctttttggtggaggaggataaaaagcacgagcgccacccaatctttggcaatatcgtgga
cgaggtggcgtaccatgaaaagtacccaaccatatatcatctgaggaagaagcttgtagacagtactg
ataaggctgacttgcggttgatctatctcgcgctggcgcatatgatcaaatttcggggacacttcctc
atcgagggggacctgaacccagacaacagcgatgtcgacaaactctttatccaactggttcagactta
caatcagcttttcgaagagaacccgatcaacgcatccggagttgacgccaaagcaatcctgagcgcta
ggctgtccaaatcccggcggctcgaaaacctcatcgcacagctccctggggagaagaagaacggcctg
tttggtaatcttatcgccctgtcactcgggctgacccccaactttaaatctaacttcgacctggccga
agatgccaagcttcaactgagcaaagacacctacgatgatgatctcgacaatctgctggcccagatcg
gcgaccagtacgcagacctttttttggcggcaaagaacctgtcagacgccattctgctgagtgatatt
ctgcgagtgaacacggagatcaccaaagctccgctgagcgctagtatgatcaagcgctatgatgagca
ccaccaagacttgactttgctgaaggcccttgtcagacagcaactgcctgagaagtacaaggaaattt
tcttcgatcagtctaaaaatggctacgccggatacattgacggcggagcaagccaggaggaattttac
aaatttattaagcccatcttggaaaaaatggacggcaccgaggagctgctggtaaagcttaacagaga
agatctgttgcgcaaacagcgcactttcgacaatggaagcatcccccaccagattcacctgggcgaac
tgcacgctatcctcaggcggcaagaggatttctacccctttttgaaagataacagggaaaagattgag
aaaatcctcacatttcggataccctactatgtaggccccctcgcccggggaaattccagattcgcgtg
gatgactcgcaaatcagaagagaccatcactccctggaacttcgaggaagtcgtggataagggggcct
ctgcccagtccttcatcgaaaggatgactaactttgataaaaatctgcctaacgaaaaggtgcttcct
aaacactctctgctgtacgagtacttcacagtttataacgagctcaccaaggtcaaatacgtcacaga
agggatgagaaagccagcattcctgtctggagagcagaagaaagctatcgtggacctcctcttcaaga
cgaaccggaaagttaccgtgaaacagctcaaagaagactatttcaaaaagattgaatgtttcgactct
gttgaaatcagcggagtggaggatcgcttcaacgcatccctgggaacgtatcacgatctcctgaaaat
cattaaagacaaggacttcctggacaatgaggagaacgaggacattcttgaggacattgtcctcaccc
ttacgttgtttgaagatagggagatgattgaagaacgcttgaaaacttacgctcatctcttcgacgac
aaagtcatgaaacagctcaagaggcgccgatatacaggatgggggcggctgtcaagaaaactgatcaa
tgggatccgagacaagcagagtggaaagacaatcctggattttcttaagtccgatggatttgccaacc
ggaacttcatgcagttgatccatgatgactctctcacctttaaggaggacatccagaaagcacaagtt
tctggccagggggacagtcttcacgagcacatcgctaatcttgcaggtagcccagctatcaaaaaggg
aatactgcagacccttaaggtcgtggatgaactcgtcaaagtaatgggaaggcataagcccgagaata
tcgttatcgagatggcccgagagaaccaaactacccagaagggacagaagaacagtagggaaaggatg
aagaggattgaagagggtataaaagaactggggtcccaaatccttaaggaacacccagttgaaaacac
ccagcttcagaatgagaagctctacctgtactacctgcagaacggcagggacatgtacgtggatcagg
aactggacatcaatcggctctccgactacgacgtggatgccatcgtgccccagtcttttctcaaagat
gattctattgataataaagtgttgacaagatccgataaaaatagagggaagagtgataacgtcccctc
agaagaagttgtcaagaaaatgaaaaattattggcggcagctgctgaacgccaaactgatcacacaac
ggaagttcgataatctgactaaggctgaacgaggtggcctgtctgagttggataaagccggcttcatc
aaaaggcagcttgttgagacacgccagatcaccaagcacgtggcccaaattctcgattcacgcatgaa
caccaagtacgatgaaaatgacaaactgattcgagaggtgaaagttattactctgaagtctaagctgg
tctcagatttcagaaaggactttcagttttataaggtgagagagatcaacaattaccaccatgcgcat
gatgcctacctgaatgcagtggtaggcactgcacttatcaaaaaatatcccaagcttgaatctgaatt
tgtttacggagactataaagtgtacgatgttaggaaaatgatcgcaaagtctgagcaggaaataggca
aggccaccgctaagtacttcttttacagcaatattatgaattttttcaagaccgagattacactggcc
aatggagagattcggaagcgaccacttatcgaaacaaacggagaaacaggagaaatcgtgtgggacaa
gggtagggatttcgcgacagtccggaaggtcctgtccatgccgcaggtgaacatcgttaaaaagaccg
aagtacagaccggaggcttctccaaggaaagtatcctcccgaaaaggaacagcgacaagctgatcgca
cgcaaaaaagattgggaccccaagaaatacggcggattcgattctcctacagtcgcttacagtgtact
ggttgtggccaaagtggagaaagggaagtctaaaaaactcaaaagcgtcaaggaactgctgggcatca
caatcatggagcgatcaagcttcgaaaaaaaccccatcgactttctcgaggcgaaaggatataaagag
gtcaaaaaagacctcatcattaagcttcccaagtactctctctttqagcttgaaaacggccggaaacg
aatgctcgctagtgcgggcgagctgcagaaaggtaacgagctggcactgccctctaaatacgttaatt
tcttgtatctggccagccactatgaaaagctcaaagggtctcccgaagataatgagcagaagcagctg
ttcgtggaacaacacaaacactaccttgatgagatcatcgagcaaataagcgaattctccaaaagagt
gatcctcgccgacgctaacctcgataaggtgctttctgcttacaataagcacagggataagcccatca
gggagcaggcagaaaacattatccacttgtttactctgaccaacttgggcgcgcctgcagccttcaag
tacttcgacaccaccatagacagaaagcggtacacctctacaaaggaggtcctggacgccacactgat
tcatcagtcaattacggggctctatgaaacaagaatcgacctctctcagctcggtggagacagcaggg
ctgaccccaagaagaagaggaaggtggctagcgatgctaagtcactgactgcctggtcccggacactg
gtgaccttcaaggatgtgtttgtggacttcaccagggaggagtggaagctgctggacactgctcagca
gatcctgtacagaaatgtgatgctggagaactataagaacctggtttccttgggttatcagcttacta
agccagatgtgatcctccggttggagaagggagaagagccctggctggtggagagagaaattcaccaa
gagacccatcctgattcagagactgcatttgaaatcaaatcatcagttccgaaaaagaaacgcaaagt
ttga
SEQ ID NO: 46
Streptococcus pyogenes dCas9-KRAB protein sequence
MDYKDHDGDYKDHDIDYKDDDDKMAPKKKRKVGRGMDKKYSIGLAIGTNSVGWAVITDEYKVPSKKFK
VLGNTDRHSIKKNLIGALLFDSGETAEATRLKRTARRRYTRRKNRICYLQEIFSNEMAKVDDSFFHRL
EESELVEEDKKHERHPIFGNIVDEVAYHEKYPTIYHLRKKLVDSTDKADLRLIYLALAHMIKERGHFL
IEGDLNPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPELKDNREKIE
KILTFRIPYYVGPLARGNSREAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFKKIECFDS
VEISGVEDRENASLGTYHDLLKIIKDKDFLDNEENEDILEDIVLTLTLFEDREMIEERLKTYAHLFDD
KVMKQLKRRRYTGWGRLSRKLINGIRDKQSGKTILDELKSDGFANRNEMQLIHDDSLTFKEDIQKAQV
SGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLETLTNLGAPAAFK
YFDTTIDRKRYTSTKEVLDATLIHQSITGLYETRIDLSQLGGDSRADPKKKRKVASDAKSLTAWSRTL
VTFKDVEVDFTREEWKLLDTAQQILYRNVMLENYKNLVSLGYQLTKPDVILRLEKGEEPWLVEREIHQ
ETHPDSETAFEIKSSVPKKKRKV
SEQ ID NO: 47
Staphylococcus aureus dCas9-KRAB polynucleotide sequence
atggccccaaagaagaagcggaaggtcggtatccacggagtcccagcagccaagcggaactacatcct
gggcctggccatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaagccagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaagggatccgat
gctaagtcactgactgcctggtcccggacactggtgaccttcaaggatgtgtttgtggacttcaccag
ggaggagtggaagctgctggacactgctcagcagatcctgtacagaaatgtgatgctggagaactata
agaacctggtttccttgggttatcagcttactaagccagatgtgatcctccggttggagaagggagaa
gagccctggctggtggagagagaaattcaccaagagacccatcctgattcagagactgcatttgaaat
caaatcatcagttccgaaaaagaaacgcaaagtt
SEQ ID NO: 48
Staphylococcus aureus dCas9-KRAB protein sequence
MAPKKKRKVGIHGVPAAKRNYILGLAIGITSVGYGIIDYETRDVIDAGVRLFKEANVENNEGRRSKRG
ARRLKRRRRHRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYAYNADLY
NALNDLNNLVITRDENEKLEYYEKFQIIENVEKQKKKPTLKQIAKEILVNEEDIKGYRVTSTGKPEFT
NLKVYHDIKDITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVKRSFIQSIKV
INAIIKKYGLPNDIIIELAREKNSKDAQKMINEMQKRNRQTNERIEEIIRTTGKENAKYLIEKIKLHD
MQEGKCLYSLEAIPLEDLLNNPFNYEVDHIIPRSVSFDNSENNKVLVKQEEASKKGNRTPFQYLSSSD
SKISYETFKKHILNLAKGKGRISKTKKEYLLEERDINRFSVQKDEINRNLVDTRYATRGLMNLLRSYE
RVNNLDVKVKSINGGFTSFLRRKWKFKKERNKGYKHHAEDALIIANADFIFKEWKKLDKAKKVMENQM
FEEKQAESMPEIETEQEYKEIFITPHQIKHIKDEKDYKYSHRVDKKPNRELINDTLYSTRKDDKGNTL
IVNNLNGLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKEVTVKNLDVIKK
ENYYEVNSKCYEEAKKLKKISNQAEFIASFYNNDLIKINGELYRVIGVNNDLLNRIEVNMIDITYREY
LENMNDKRPPRIIKTIASKTQSIKKYSTDILGNLYEVKSKKHPQIIKKGKRPAATKKAGQAKKKKGSD
AKSLTAWSRTLVTFKDVFVDFTREEWKLLDTAQQILYRNVMLENYKNLVSLGYQLTKPDVILRLEKGE
EPWLVEREIHQETHPDSETAFEIKSSVPKKKRKV
SEQ ID NO: 49
Tet1CD polynucleotide sequence
ctgcccacctgcagctgtcttgatcgagttatacaaaaagacaaaggcccatattatacacaccttgg
ggcaggaccaagtgttgctgctgtcagggaaatcatggagaataggtatggtcaaaaaggaaacgcaa
taaggatagaaatagtagtgtacaccggtaaagaagggaaaagctctcatgggtgtccaattgctaag
tgggttttaagaagaagcagtgatgaagaaaaagttctttgtttggtccggcagcgtacaggccacca
ctgtccaactgctgtgatggtggtgctcatcatggtgtgggatggcatccctcttccaatggccgacc
ggctatacacagagctcacagagaatctaaagtcatacaatgggcaccctaccgacagaagatgcacc
ctcaatgaaaatcgtacctgtacatgtcaaggaattgatccagagacttgtggagcttcattctcttt
tggctgttcatggagtatgtactttaatggctgtaagtttggtagaagcccaagccccagaagattta
gaattgatccaagctctcccttacatgaaaaaaaccttgaagataacttacagagtttggctacacga
ttagctccaatttataagcagtatgctccagtagcttaccaaaatcaggtggaatatgaaaatgttgc
ccgagaatgtcggcttggcagcaaggaaggtcgacccttctctggggtcactgcttgcctggacttct
gtgctcatccccacagggacattcacaacatgaataatggaagcactgtggtttgtaccttaactcga
gaagataaccgctctttgggtgttattcctcaagatgagcagctccatgtgctacctctttataagct
ttcagacacagatgagtttggctccaaggaaggaatggaagccaagatcaaatctggggccatcgagg
tcctggcaccccgccgcaaaaaaagaacgtgtttcactcagcctgttccccgttctggaaagaagagg
gctgcgatgatgacagaggttcttgcacataagataagggcagtggaaaagaaacctattccccgaat
caagcggaagaataactcaacaacaacaaacaacagtaagccttcgtcactgccaaccttagggagta
acactgagaccgtgcaacctgaagtaaaaagtgaaaccgaaccccattttatcttaaaaagttcagac
aacactaaaacttattcgctgatgccatccgctcctcacccagtgaaagaggcatctccaggcttctc
ctggtccccgaagactgcttcagccacaccagctccactgaagaatgacgcaacagcctcatgcgggt
tttcagaaagaagcagcactccccactgtacgatgccttcgggaagactcagtggtgccaatgctgca
gctgctgatggccctggcatttcacagcttggcgaagtggctcctctccccaccctgtctgctcctgt
gatggagcccctcattaattctgagccttccactggtgtgactgagccgctaacgcctcatcagccaa
accaccagccctccttcctcacctctcctcaagaccttgcctcttctccaatggaagaagatgagcag
cattctgaagcagatgagcctccatcagacgaacccctatctgatgaccccctgtcacctgctgagga
gaaattgccccacattgatgagtattggtcagacagtgagcacatctttttggatgcaaatattggtg
gggtggccatcgcacctgctcacggctcggttttgattgagtgtgcccggcgagagctgcacgctacc
actcctgttgagcaccccaaccgtaatcatccaacccgcctctcccttgtcttttaccagcacaaaaa
cctaaataagccccaacatggttttgaactaaacaagattaagtttgaggctaaagaagctaagaata
agaaaatgaaggcctcagagcaaaaagaccaggcagctaatgaaggtccagaacagtcctctgaagta
aatgaattgaaccaaattccttctcataaagcattaacattaacccatgacaatgttgtcaccgtgtc
cccttatgctctcacacacgttgcggggccctataaccattgggtc
SEQ ID NO: 50
Tet1CD amino acid sequence
LPTCSCLDRVIQKDKGPYYTHLGAGPSVAAVREIMENRYGQKGNAIRIEIVVYTGKEGKSSHGCPIAK
WVLRRSSDEEKVLCLVRQRTGHHCPTAVMVVLIMVWDGIPLPMADRLYTELTENLKSYNGHPTDRRCT
LNENRICICQGIDPETCGASFSFGCSWSMYFNGCKFGRSPSPRRFRIDPSSPLHEKNLEDNLQSLATR
LAPIYKQYAPVAYQNQVEYENVARECRLGSKEGRPFSGVTACLDFCAHPHRDIHNMNNGSTVVCTLTR
EDNRSLGVIPQDEQLHVLPLYKLSDTDEFGSKEGMEAKIKSGAIEVLAPRRKKRTCFTQPVPRSGKKR
AAMMTEVLAHKIRAVEKKPIPRIKRKNNSTTTNNSKPSSLPTLGSNTETVQPEVKSETEPHFILKSSD
NTKTYSLMPSAPHPVKEASPGFSWSPKTASATPAPLKNDATASCGFSERSSTPHCTMPSGRLSGANAA
AADGPGISQLGEVAPLPTLSAPVMEPLINSEPSTGVTEPLTPHQPNHQPSFLTSPQDLASSPMEEDEQ
HSEADEPPSDEPLSDDPLSPAEEKLPHIDEYWSDSEHIFLDANIGGVAIAPAHGSVLIECARRELHAT
TPVEHPNRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWV
SEQ ID NO: 51
DNA sequence of the gRNA constant region
gtttaagagctatgctggaaacagcatagcaagtttaaataaggctagtccgttatcaactt
gaaaaagtggcaccgagtcggtgc
SEQ ID NO: 52
RNA sequence of the gRNA constant region
guuuaagagcuaugcuggaaacagcauagcaaguuuaaauaaggcuaguccquuaucaacuu
gaaaaaguggcaccgagucggugc
SEQ ID NO: 53
JCR 179 DNA
AACACACAGCIGGGTTATCAGAG
SEQ ID NO: 54
JCR183 DNA
GAACTGGTGGGAAATGGTCTAG
SEQ ID NO: 103
JCR 179 RNA
AACACACAGCUGGGUUAUCAGAG
SEQ ID NO: 104
JCR183 RNA
GAACUGGUGGGAAAUGGUCUAG

1. A method of screening for a pair of gRNA molecules for editing a genomic nucleic acid in a subject, the method comprising:
(a) generating a plurality of pairs of gRNA molecules, each pair comprising a first gRNA and a second gRNA, wherein the first gRNA targets a first nucleic acid sequence and the second gRNA targets a second nucleic acid sequence;
(b) expressing a Cas9 protein or a fusion protein comprising the Cas9 protein, and the plurality of pairs of gRNA molecules in a plurality of cells, wherein one pair of gRNA molecules is expressed in a cell, and wherein the first gRNA directs the Cas9 protein or fusion protein to cut the first nucleic acid sequence and the second gRNA directs the Cas9 protein or fusion protein to cut the second nucleic acid sequence.

2. The method of claim 1, wherein expressing the Cas9 protein or the fusion protein comprising the Cas9 protein, and the plurality of pairs of gRNA molecules in the plurality of cells, wherein one pair of gRNA molecules is expressed in a cell, and wherein the first gRNA directs the Cas9 protein or fusion protein to cut the first nucleic acid sequence and the second gRNA directs the Cas9 protein or fusion protein to cut the second nucleic acid sequence in step (b), thereby forms an excised nucleic acid and a new junction in the genomic nucleic acid.

3. The method of claim 2, wherein the excised nucleic acid is in-frame.

4. The method of any one of claims 1-3, wherein the genomic nucleic acid comprises at least one exon of a dystrophin gene,
wherein the first nucleic acid sequence comprises a first intron of the dystrophin gene and the second nucleic acid sequence comprises a second intron of the dystrophin gene, and
wherein the first intron is adjacent to one side of the at least one exon and the second intron is adjacent to the other side of the at least one exon.

5. The method of claim 4, wherein the at least one exon is in between the first and second introns in the genomic nucleic acid.

6. The method of any one of claims 1-5, wherein the genomic nucleic acid comprises two or more exons of a dystrophin gene,
wherein the first nucleic acid sequence comprises a first intron of the dystrophin gene and the second nucleic acid sequence comprises a second intron of the dystrophin gene, and
wherein the first intron is adjacent to one side of the two or more exons and the second intron is adjacent to the other side of the two or more exons.

7. The method of claim 6, wherein the two or more exons are in between the first and second introns in the genomic nucleic acid.

8. The method of any one of claims 1-7, wherein the expression is effected by transfecting the plurality of cells with a plurality of vectors, wherein each cell is transfected with a first vector encoding one pair of gRNA molecules and a second vector encoding the Cas9 protein or fusion protein, wherein each cell is transfected with a different first vector encoding a different pair of gRNA molecules.

9. The method of claim 8, wherein the first vector and second vector are each a viral vector.

10. The method of claim 9, wherein the viral vector is a lentiviral vector, a AAV vector, or an adenoviral vector.

11. The method of any one of claims 1-10, the method further comprising:
(c) isolating the genomic nucleic acid from the plurality of cells; and/or
(d) contacting the genomic nucleic acid with a first pool of probes, wherein one or more different probes specifically bind to each new junction and a portion of the first nucleic acid sequence; and/or
(e) isolating the genomic nucleic acid bound to the first pool of probes; and/or
(f) contacting the genomic nucleic acid bound to the first pool of probes with a second pool of probes, wherein one or more different probes specifically bind to each new junction and a portion of the second nucleic acid sequence; and/or
(g) isolating the genomic nucleic acid bound to the first and second pools of probes; and/or
(h) sequencing the isolated genomic nucleic acid bound to the first and second pools of probes; and/or
(i) aligning the sequenced isolated genomic nucleic acid to identify the sequenced new junctions; and/or
(j) assigning each sequenced new junction to the corresponding pair of gRNA molecules.

12. The method of claim 11, wherein step (i) comprises computationally aligning the sequences of the isolated genomic nucleic acid to identify the sequenced new junctions.

13. The method of claim 12 or 13, further comprising identifying the pair of gRNA molecules having a greater number of sequenced new junctions as the pair of gRNA molecules having greater efficiency.

14. The method of any one of claims 11-13, wherein the probes each have a length of about 100 bp to about 140 bp.

15. The method of any one of claims 1-14, wherein the excised nucleic acid comprises exon 51 of the dystrophin gene.

16. The method of any one of claims 1-15, wherein the excised nucleic acid comprises exons 45-55 of the dystrophin gene.

17. The method of any one of claims 1-15, wherein the first nucleic acid sequence is within intron 50 of the dystrophin gene.

18. The method of any one of claims 1-15, wherein the second nucleic acid sequence is within intron 51 of the dystrophin gene.

19. The method of any one of claims 1-16, wherein the first nucleic acid sequence is within intron 44 of the dystrophin gene.

20. The method of any one of claims 1-16, wherein the second nucleic acid sequence is within intron 55 of the dystrophin gene.

21. The method of any one of claims 1-20, wherein the probes are biotinylated probes.

22. A pair of gRNA molecules identified by the method of any one of the preceding claims.

23. A CRISPR/Cas9 system comprising the pair of gRNA molecules of claim 22.

24. A gRNA molecule that binds and targets a polynucleotide sequence, and wherein the gRNA molecule binds or is encoded by a polynucleotide comprising a sequence selected from SEQ ID NOs: 55-78, or wherein the gRNA molecule comprises a polynucleotide sequence selected from SEQ ID NOs: 79-102.

25. A transgenic mouse whose genome comprises:
a mutation in the mouse dystrophin gene;
a mutant human dystrophin gene on chromosome 5; and
a mutation in the mouse utrophin gene.

26. The mouse of claim 25, wherein the mutation in the mouse dystrophin gene comprises an insertion or deletion in the mouse dystrophin gene that prevents protein expression from the mouse dystrophin gene.

27. The mouse of claim 26, wherein the mutation in the mouse dystrophin gene comprises a premature stop codon in exon 23 of the mouse dystrophin gene.

28. The mouse of any one of claims 25-27, wherein the mutant human dystrophin gene has at least one exon deleted.

29. The mouse of any one of claims 25-28, wherein the mutant human dystrophin gene has exon 52 deleted.

30. The mouse of any one of claims 25-29, wherein the mutation in the mouse utrophin gene is a functional deletion of the mouse utrophin gene.

31. The mouse of any one of claims 25-29, wherein the mutation in the mouse utrophin gene comprises an insertion or deletion in the mouse utrophin gene that prevents protein expression from the mouse utrophin gene.

32. The mouse of claim 31, wherein the mutation in the mouse utrophin gene comprises an insertion in exon 7 of the mouse utrophin gene.

33. The mouse of any one of claims 25-29, wherein the mutation in the mouse utrophin gene comprises a deletion of the entire mouse utrophin gene.

34. The mouse of any one of claims 25-33, wherein the mouse is heterozygous for the mutation in the mouse utrophin gene.

35. The mouse of any one of claims 25-33, wherein the mouse is homozygous for the mutation in the mouse utrophin gene.

36. The mouse of any one of claims 25-35, wherein the mouse has reduced life span, reduced body mass, reduced body strength, reduced motor coordination, reduced balance, and/or reduced forelimb strength as compared to a wild-type mouse.

37. The mouse of any one of claims 25-35, wherein the mouse has reduced life span, reduced body mass, reduced body strength, reduced motor coordination, reduced balance, and/or reduced forelimb strength as compared to a control mouse whose genome comprises a wild-type utrophin gene and a mutation in the mouse dystrophin gene.

38. The mouse of any one of claims 25-35, wherein the mouse has reduced lifespan, reduced body mass, reduced body strength, reduced motor coordination, reduced balance, and/or reduced forelimb strength as compared to a control mouse whose genome comprises a wild-type utrophin gene, a mutation in the mouse dystrophin gene, and a mutant human dystrophin gene.

39. The mouse of any of claims 25-38, wherein the mouse has increased muscle damage as compared to (i) a wild-type mouse, (ii) a control mouse whose genome comprises a wild-type utrophin gene and a mutation in the mouse dystrophin gene, and/or (iii) a control mouse whose genome comprises a wild-type utrophin gene, a mutation in the mouse dystrophin gene, and a mutant human dystrophin gene.

40. The mouse of claim 39, wherein the muscle damage comprises one or more of degeneration of the muscle, fibrosis of the muscle, and elevated serum creatine kinase.

41. The mouse of any one of claims 25-40, wherein the mouse does not exhibit detectable dystrophin protein in heart or skeletal muscle.

42. The mouse of any one of claims 25-41, wherein the mouse is a hDMDΔ52/mdx/Utrn KO mouse.

43. An isolated cell or biological material obtained from the mouse of any one of claims 25-42.

44. The biological material of claim 43, comprising a protein, a lipid, a nucleotide, fat, muscle, or a tissue.

45. A method of correcting a dystrophin gene mutation, the method comprising administering to the mouse of any one of claims 25-42 a CRISPR/Cas9 gene editing composition.

46. The method of claim 45, wherein the CRISPR/Cas9 gene editing composition comprises:
(a) at least one guide RNA (gRNA) targeting the mutant human dystrophin gene; and
(b) a Cas9 protein or a fusion protein comprising the Cas9 protein.

47. The method of claim 46, wherein the CRISPR/Cas9 gene editing composition comprises a first gRNA and a second gRNA, and wherein the first gRNA and the second gRNA are configured to form a first and a second double strand break in a first and a second intron flanking exon 51 of the mutant human dystrophin gene, respectively, thereby deleting exon 51.

48. The method of claim 47, wherein the CRISPR/Cas9 gene editing composition comprises a first gRNA and a second gRNA, and wherein the first gRNA and the second gRNA are configured to form a first and a second double strand break in a first and a second intron flanking exons 45-55 of the mutant human dystrophin gene, respectively, thereby deleting exons 45-55.

49. The method of any one of claims 45-48, wherein the dystrophin gene mutation is corrected in a cell of the mouse, and wherein the cell is a muscle cell, a satellite cell, or an iPSC/iCM.

50. The method of any one of claims 45-49, wherein the correction restores the reading frame of the human dystrophin gene.

51. The method of any one of claims 45-50, wherein the correction results in expression of an at least partially functional human dystrophin protein.

52. A gamete produced by the mouse of any one of claims 25-42.

53. The gamete of claim 52, wherein the gamete does not encode a functional mouse dystrophin protein or a functional mouse utrophin protein.

54. An isolated mouse cell, or a progeny cell thereof, isolated from the mouse of any one of claims 25-42.

55. A primary cell culture or a secondary cell line derived from the mouse of any one of claims 25-42.

56. A tissue or organ explant or culture thereof, derived from the mouse of any one of claims 25-42

57. A method of screening therapeutic agents for treating Duchenne muscular dystrophy (DMD), the method comprising administering to the mouse of any one of claims 25-42 one or more therapeutic agents.

58. The method of claim 57, wherein the one or more therapeutic agents comprises a small molecule, anti-sense RNA, vector, CRISPR/Cas gene editing system, or biological agent, or a combination thereof.

59. The method of claim 58, wherein the vector is a viral vector encoding a gene of interest.

60. The method of claim 59, wherein the viral vector is an AAV vector.

61. The method of any one of claims 57-60, wherein the mouse after administration of the one or more therapeutic agents exhibits increased lifespan, reduced body mass, increased body strength, increased motor coordination, increased balance, increased forelimb strength, reduced muscle injury, and/or reduced CK level compared to before administration of the one or more therapeutic agents.

62. The method of any one of claims 57-61, wherein the mouse after administration of the one or more therapeutic agents exhibits increased expression of a dystrophin gene as compared to before administration of the one or more therapeutic agents.

63. The method of claim 62, wherein the dystrophin gene is a truncated human dystrophin gene.

64. The method of claim 63, wherein the truncated human dystrophin gene comprises a plurality of deletions relative to a wild-type human dystrophin gene, and wherein at least one of the deletions is in exon 52.
